(19) United States
(12) Patent Application Publication
(10) Pub. No.: US 2022/0199239 A1
(43) Pub. Date: Jun. 23, 2022

(54) STORAGE MANAGEMENT DEVICE FOR MEDICAL INSTRUMENT, STORAGE MANAGEMENT SYSTEM, AND STORAGE MANAGEMENT METHOD FOR MEDICAL INSTRUMENT

(71) Applicant: DGSHAPE Corporation, Hamamatsu-shi (JP)

(72) Inventors: Akinori SUGAYA, Hamamatsu-shi (JP); Masaki HANAJIMA, Hamamatsu-shi (JP); Akira OTAKA, Hamamatsu-shi (JP); Takaaki KOKUBO, Hamamatsu-shi (JP)

(21) Appl. No.: 17/558,672

(22) Filed: Dec. 22, 2021

(30) Foreign Application Priority Data
Dec. 23, 2020 (JP) 2020-214046

(51) Int. Cl. G16H 40/20; G16H 40/40

(52) U.S. Cl. CPC G16H 40/20; G16H 40/40

(57) ABSTRACT

A storage management device stores sterilization information in which a sterilization ID of an instrument set and a rack ID of a rack number of a rack are associated with one another. A target rack ID assigned to the rack number of the rack in which the instrument set is stored is acquired. A target sterilization ID of the instrument set that is stored is acquired. The rack ID associated with the sterilization ID that matches the target sterilization ID is extracted from the sterilization information and the rack ID is determined as an extracted rack ID. Whether the extracted rack ID matches the target rack ID is determined. When the extracted rack ID does not match the target rack ID, a wrong rack is notified.

### CROSS REFERENCE TO RELATED APPLICATIONS

This application claims the benefit of priority to Japanese Patent Application No. 2020-214046, filed on Dec. 23, 2020. The entire contents of this application are incorporated herein by reference.

### BACKGROUND OF THE INVENTION

### 1. Field of the Invention

The present invention relates to a storage management device for a medical instrument, a storage management system, and a storage management method for a medical instrument.

### 2. Description of the Related Art

For example, Japanese Laid-open Patent Publication No. 2017-148113 discloses a medical instrument repeatedly used in a medical practice, such as a surgical operation, a medical examination, or the like. The medical instrument can be repeatedly used by sequentially performing predetermined medical treatment processes. The medical treatment processes include a surgical operation process in which a surgical operation is performed, a collection process, a washing process, an assembling process, a sterilization process, and a storage process.

In the collection process, a medical instrument is collected after a surgical operation. In the washing process, the collected medical instrument is disassembled into a plurality of separate components and the separate components are washed. In the assembling process, the components of the medical instrument that have been washed are assembled. In the sterilization process, sterilization processing is performed on the medical instrument that has been assembled. In the storage process, the sterilized medical instrument is stored in a predetermined storage area. In the surgical operation process, the medical instrument stored in a storage area is dispensed and a surgical operation is performed using the dispensed medical instrument.

Incidentally, in the storage process, the medical instrument is stored, for example, in a predetermined rack. For example, the rack is divided to a plurality of areas and a rack number is assigned to each area. For the medical instrument, in which rack number of which rack the medical instrument is stored is set in advance. However, storage of the medical instrument is manually performed by an operator, and therefore, there is a probability that, in storing the medical instrument, there can be some operators who do not store the medical instrument in the rack number of the rack set in advance but store the medical instrument in a wrong storage destination in some cases.

### SUMMARY OF THE INVENTION

Preferred embodiments of the present invention provide storage management devices for medical instruments, storage management systems, and storage management methods for medical instruments, which each prevent in advance an operator erroneously storing a medical instrument in a wrong storage destination.

A storage management device for a medical instrument according to a preferred embodiment of the present invention includes a memory and at least one processor. The storage management device stores storage destination information in which at least an identification ID used to identify an instrument set including one or more medical instruments and a rack ID assigned to a rack number of a rack in which the instrument set is to be stored are associated with one another. The at least one processor is configured to execute a program stored in the memory to perform rack ID acquisition processing, instrument ID acquisition processing, rack ID extraction processing, rack determination processing, and wrong rack notification processing. In the rack ID acquisition processing, a target rack ID assigned to the rack number of the rack in which the instrument set is to be stored is acquired. In the instrument ID acquisition processing, a target identification ID used to identify the instrument set that is to be stored is acquired. In the rack ID extraction processing, the rack ID associated with the identification ID that matches the target identification ID is extracted from the storage destination information and the rack ID is determined as an extracted rack ID. In the rack determination processing, whether the extracted rack ID matches the target rack ID is determined. In the wrong rack notification processing, when it is determined in the rack determination processing that the extracted rack ID does not match the target rack ID, a wrong rack of the instrument set that is identified from the target identification ID is notified.

According to the storage management device for a medical instrument according to a preferred embodiment of the present invention, in the storage destination information, the rack ID assigned to the rack number of the rack of a proper storage destination of the instrument set that is identified from the identification ID is associated with the identification ID. Herein, in storing the instrument set, an operator extracts the rack ID corresponding to the target identification ID of the instrument set that the operator is going to store from the storage destination information to determine the rack ID as an extracted rack ID. Then, whether the target rack ID assigned to the rack number of the rack in which the operator is going to store the instrument set and the extracted rack ID match one another is determined. Herein, if the target rack ID does not match the extracted rack ID, it is determined that the rack number of the rack in which the operator is going to store the instrument set is not proper and a wrong rack is notified. The operator is notified of a wrong rack, and thus, can notice a wrong rack before storing the instrument set. Accordingly, an error in which an operator erroneously stores an instrument set in a wrong storage destination is prevented in advance.

In a storage management method for a medical instrument according to a preferred embodiment of the present invention, storage destination information in which at least an identification ID used to identify an instrument set including one or more medical instruments and a rack ID assigned to a rack number of a rack in which the instrument set is to be stored are associated with one another is stored. The storage management method includes a rack ID acquisition step, an instrument identification step, a rack ID extraction step, a rack determination step, and a wrong rack notification step. In the rack ID acquisition step, a target rack ID assigned to the rack number of the rack in which the instrument set is to be stored is acquired. In the instrument identification step, a target identification ID used to identify the instrument set that is to be stored is acquired. In the rack ID extraction step, the rack ID associated with the identification ID that matches the target identification ID is extracted from the storage destination information and the rack ID is determined as an extracted rack ID. In the rack determination step, whether the extracted rack ID matches the target rack ID is determined. In the wrong rack notification step, when it is determined in the rack determination processing that the extracted rack ID does not match the target rack ID, a wrong rack of the instrument set that is identified from the target identification ID is notified.

The above and other elements, features, steps, characteristics and advantages of the present invention will become more apparent from the following detailed description of the preferred embodiments with reference to the attached drawings.

### BRIEF DESCRIPTION OF THE DRAWINGS

FIG. 1 is a schematic view illustrating a storage management system according to a preferred embodiment of the present invention.

FIG. 2 is a diagram illustrating a circulation cycle.

FIG. 3 is a block diagram of a storage management device.

FIG. 4 is a table illustrating a configuration example of an instrument set master table.

FIG. 5 is a table illustrating a configuration example of a rack master table.

FIG. 6 is a table illustrating a configuration example of a sterilization table.

FIG. 7 is a table illustrating a configuration example of a storage history table.

FIG. 8A is a flowchart illustrating process steps of processing of the storage management device in stocking an instrument set.

FIG. 8B is a flowchart illustrating process steps of processing of the storage management device in stocking an instrument set.

FIG. 9 is a view illustrating a stock registration screen.

FIG. 10 is a view illustrating a wrong rack warning screen.

FIG. 11A is a view illustrating an unsterilized warning screen.

FIG. 11B is a view illustrating a sterilization validity expiration warning screen.

FIG. 12 is a view illustrating a re-stocking warning screen.

FIG. 13 is a view illustrating a stock registration screen in a state where an instrument set has been added to a stock list.

### DETAILED DESCRIPTION OF THE PREFERRED EMBODIMENTS

With reference to the attached drawings, storage management systems each including a storage management device for a medical instrument (which will be hereinafter simply referred to as a storage management device) according to preferred embodiments of the present invention will be described below. Note that, as a matter of course, preferred embodiments described herein are not intended to be particularly limiting of the present invention.

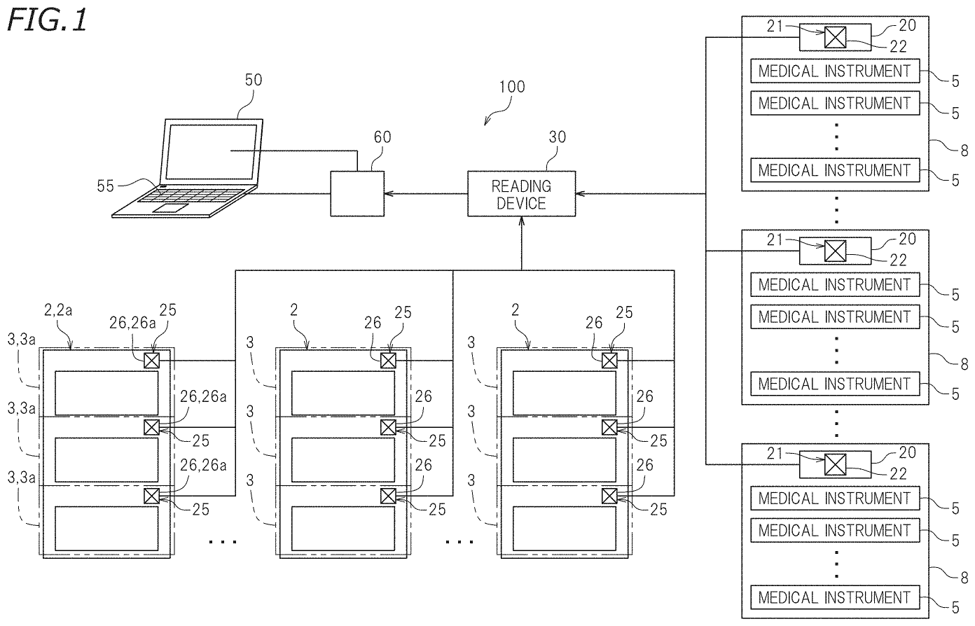

FIG. 1 is a schematic view illustrating a storage management system 100 according to a preferred embodiment of the present invention. The storage management system 100 is used in storing a medical instrument 5 in a rack number 3 of a predetermined rack 2.

The medical instrument 5 is an instrument that is used in a medical practice that is performed in a medical site, such as a hospital or the like. The medical practice is a practice related to a medical activity that is performed for a patient by a doctor. Examples of a medical practice include a surgical operation and a medical examination.

The medical instrument 5 is, for example, a trocar, forceps, an incision device, a washing and sucking device, scissors, a scalpel (for example, a laser scalpel or an electric scalpel), an ultrasonic cutter, a scalpel holder, a cannula, tweezers, a retractor, a scale, a sonde, an elevator, a rasp, a suction tube, a rib retractor, a rib contractor, a needle holder, an injector, a metal ball, a pus tray, a cup, a pin, a mirror, a file, an opening tool, a clamp, a handpiece, an elevatorium, a chisel, a sharp spoon, a raspatory, a speculum, a suture needle, a punch, a water receiver, a needle, a penetrator, a bougie, a vent pipe, a bone impactor, a luer, a radio plier, a hammer, an angle gauge, a thermometer, a perforator, a spuit, a metal swab, an enema, a syringe, an endoscope, or the like. However, the above-described examples are merely some examples of the medical instrument 5. The medical instrument 5 is not limited to the above-described examples.

The medical instrument 5 may be formed of a single part and may be formed of a plurality of parts. Examples of the medical instrument 5 formed of a plurality of parts include, for example, a trocar, forceps, an incision device, a washing and sucking device, or the like which is used in a laparoscopic surgical operation. The medical instrument 5 described above is formed of a plurality of components, is collected, for example, after a surgical operation, and is disassembled into a plurality of separate components. Parts that form the medical instrument 5 are referred to as components herein. Also, in some cases, such a component is further formed of a plurality of components, and in such a case, all of the components are referred to as components.

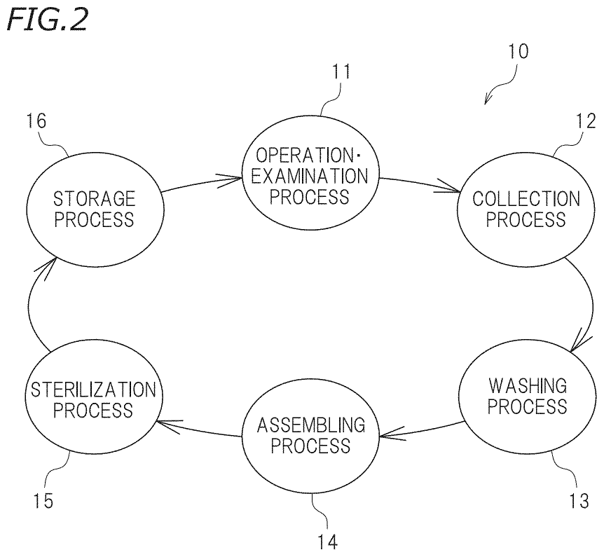

FIG. 2 is an explanatory diagram of a circulation cycle 10. As illustrated in FIG. 2, the medical instrument 5 is repeatedly usable in a predetermined circulation cycle 10. Note that the number of repetitions of use of one medical instrument 5 in the circulation cycle 10 is not particularly limited and is determined in accordance with a type of the medical instrument 5 or a component that forms the medical instrument 5, as appropriate. In this preferred embodiment, the circulation cycle 10 includes an operation and examination process 11, a collection process 12, a washing process 13, an assembling process 14, a sterilization process 15, and a storage process 16.

The operation and examination process 11 is a process in which a surgical operation or a medical examination which is an example of a medical practice is performed using the medical instrument 5. In the operation and examination process 11, the medical instrument 5 that is stored in a rack number 3 of a predetermined rack 2 (see FIG. 1) arranged in a predetermined storage area is dispensed (in other words, is taken out) and a surgical operation or a medical examination is performed using the dispensed medical instrument 5.

The collection process 12 is a process that is performed after the operation and examination process 11. The collection process 12 is a process in which the medical instrument 5 that has been used in a surgical operation or a medical examination is collected. Note that, in a case where the medical instrument 5 is formed of a plurality of components, the collection process 12 may include an operation of disassembling the medical instrument 5 into individual components.

The washing process 13 is a process that is performed after the collection process 12. The washing process 13 is a process in which the medical instrument 5 or each of the individual components of the medical instrument 5 that has been collected in the collection process 12 is washed. In the washing process 13, for example, using a washer (not illustrated), washing of the medical instrument 5 is performed.

The assembling process 14 is a process that is performed after the washing process 13. The assembling process 14 is a process in which the components of the medical instrument 5 which have been washed in the washing process 13 are assembled to complete the single medical instrument 5. The assembling process 14 is a process in which the medical instrument 5 that is formed of a plurality of components is assembled. For example, for the medical instrument 5 that is formed of a single component or the medical instrument 5 that has not been disassembled in the collection process 12, the assembling process 14 may be omitted.

The sterilization process 15 is a process that is performed after the assembling process 14. The sterilization process 15 is a process in which the medical instrument 5 that has been assembled in the assembling process 14 is sterilized. In the sterilization process 15, for example, using a sterilizer (not illustrated), sterilization processing of the medical instrument 5 is performed. Sterilization processing will be hereinafter also referred to merely as sterilization.

The storage process 16 is a process that is performed after the sterilization process 15. The storage process 16 is a process in which the medical instrument 5 on which sterilization processing has been performed is stored in the rack number 3 of the predetermined rack 2 (see FIG. 1) arranged in the predetermined storage area. Herein, storing the medical instrument 5 in the rack number 3 of the rack 2 includes putting the medical instrument 5 in an area of the rack 2 in which the rack number 3 is designated. Storing the medical instrument 5 in the rack number 3 of the predetermined rack 2 will be hereinafter referred to as stocking. Note that, after the storage process 16, the operation and examination process 11 is performed.

In the circulation cycle 10 according to this preferred embodiment, each process is performed using an instrument set 8 illustrated in FIG. 1 as a unit. Herein, the instrument set 8 is a set including one or more medical instruments 5. There is no particular limitation on a method for dividing the plurality of medical instruments 5 into one or more instrument sets 8. The number and types of the medical instruments 5 included in the instrument set 8 are determined for each medical site, such as a hospital or the like and, in some cases, are determined based on empirical rules of staff members, such as a doctor, a nurse, an operator of each process of the circulation cycle 10, or the like.

For example, the instrument set 8 is a set including one or more medical instruments 5 that are used in one medical practice (for example, a surgical operation or a medical examination). The number of instrument sets 8 used in one surgical operation or one medical examination may be one and may be plural. The one or more medical instruments 5 included in one instrument set 8 may be housed in an unillustrated tray in a state where the one or more medical instruments 5 are put and sealed in a predetermined packaging material, and thus, is stored in the storage process 16 (see FIG. 2).

In this preferred embodiment, when sterilization processing is performed on the one or more medical instruments 5 of the instrument set 8 in the sterilization process 15 illustrated in FIG. 2, a sterilization label 20 illustrated in FIG. 1 is issued. In performing sterilization processing, the one or more medical instruments 5 of the instrument set 8 are sterilized in a state of being put in the predetermined packaging material. The sterilization label 20 is attached, for example, to the predetermined packaging material described above after sterilization processing. As illustrated in FIG. 1, a sterilization identification mark 21 is assigned to the sterilization label 20. The sterilization identification mark 21 is read by a reading device 30 that will be described later. There is no particular limitation on a type of the sterilization identification mark 21. For example, the sterilization identification mark 21 may be a one-dimensional code, may be a two-dimensional code, and may be an IC tag.

For example, a target sterilization ID 22 is recorded in the sterilization identification mark 21. The target sterilization ID 22 is an ID assigned to the instrument set 8 on which sterilization processing has been performed when the sterilization processing was performed on the instrument set 8. Accordingly, with the target sterilization ID 22 assigned, it can be known that sterilization processing has been performed on the instrument set 8. The target sterilization ID 22 is a specific ID that is assigned to the instrument set 8 each time sterilization processing is performed on the instrument set 8. Therefore, the instrument set 8 can be identified from the target sterilization ID 22. In this preferred embodiment, a new target sterilization ID 22 is assigned to one instrument set 8 each time sterilization processing is performed thereon. The target sterilization ID 22 assigned to the instrument set 8 is changed each time the instrument set 8 is used in a surgical operation or a medical examination and then is sterilized. Note that, in this preferred embodiment, the target sterilization ID 22 is an example of a target identification ID according to a preferred embodiment of the present invention.

In this preferred embodiment, in the storage process 16 of FIG. 2, the one or more medical instruments 5 are stored in units of the instrument set 8. For example, as illustrated in FIG. 1, a plurality of racks 2 are arranged in the storage area where the instrument set 8 is stored. Each of the racks 2 includes a plurality of rack numbers 3. For example, each of the racks 2 is divided into a plurality of areas and a different rack number 3 is assigned to each of the areas. In this preferred embodiment, what type of instrument set 8 is stored in each rack number 3 of each rack 2 is set in advance. However, there is no particular limitation on what type of instrument set 8 is stored in which rack number 3 of which rack 2. What type of instrument set 8 is stored in which rack number 3 of which rack 2 is determined, for example, in each medical site, such as a hospital or the like and, in some cases, is determined based on empirical rules of staff members, such as a doctor, a nurse, an operator of each process of the storage process 16, or the like.

In this preferred embodiment, a rack identification mark 25 is assigned to each rack number 3 of each rack 2. The rack identification mark 25 is read by the reading device 30. There is no particular limitation on a type of the rack identification mark 25. For example, the rack identification mark 25 is read by the reading device 30. There is no particular limitation on the type of the rack identification mark 25 but, for example, the rack identification mark 25 is the same type as that of the sterilization identification mark 21. The rack identification mark 25 may be a one-dimensional code, may be a two-dimensional code, and may be an IC tag.

In the rack identification mark 25, a target rack ID 26 is recorded. The target rack ID 26 is a specific ID assigned to each rack number 3 of each rack 2. The target rack ID 26 assigned to the rack number 3 of the rack 2 is invariable. By reading the target rack ID 26, the rack number 3 of the rack 2 can be identified from the target rack ID 26.

As described above, for the instrument set 8, the processes of the circulation cycle 10 of FIG. 2 are repeatedly performed sequentially. However, depending on the instrument set 8, the order of the sterilization process 15 and the storage process 16 is switched in some cases. In such a case, the instrument set 8 for which processes of a circulation cycle in which the order of the sterilization process 15 and the storage process 16 is switched are repeatedly performed sequentially will be referred to as a sterilization-later instrument set 8a, as illustrated in FIG. 1. For the sterilization-later instrument set 8a, after the assembling process 14, the storage process 16 is performed, and then, the sterilization process 15 is performed. After sterilization processing is performed on the sterilization-later instrument set 8a in the sterilization process 15, the sterilization-later instrument set 8a is used in the operation and examination process 11. That is, the sterilization-later instrument set 8a is used in a medical practice, such as a surgical operation, a medical examination, or the like, and then, is stored in the storage process 16 without going through sterilization processing. Then, after the sterilization-later instrument set 8a is dispensed from a storage area, sterilization processing is performed on the sterilization-later instrument set 8a and the sterilization-later instrument set 8a is used in a medical practice, such as a surgical operation, a medical examination, or the like.

In this preferred embodiment, for the sterilization-later instrument set 8a, it is set in advance to store the sterilization-later instrument set 8a in a rack number 3 of an exclusive rack 2. The rack number 3 of the rack 2 in which the instrument set 8, such as the sterilization-later instrument set 8a, that is sterilized after being stored and is used in a surgical operation or a medical examination will be hereinafter referred to as a rack number 3a of a sterilization-later rack 2a. The target rack ID 26 assigned to the rack number 3a of the sterilization-later rack 2a will be referred to as a sterilization-later rack ID 26a.

Incidentally, in the storage process 16 of FIG. 2, the instrument set 8 is stored in the rack number 3 of the predetermined rack 2 that has been set in advance. However, storage of the instrument set 8 is manually performed by the operator, and therefore, there is a probability that, in storing the instrument set 8, some operators do not store the instrument set 8 in the rack number 3 of the rack 2 that has been set in advance but erroneously store the instrument set 8 in a wrong storage destination in some cases. Erroneous storage of the instrument set 8 in a wrong storage destination will be hereinafter referred to as a wrong rack.

Furthermore, in this preferred embodiment, the instrument set 8 except for the sterilization-later instrument set 8a is stored after having gone through sterilization processing. For sterilization processing, a term of validity is set, and it is preferable that the instrument set 8 for which the term of validity of sterilization processing has expired is not stored. In a conventional manner, the operator has to know whether sterilization processing has been performed on the instrument set 8, and there is a probability that some operators erroneously store the instrument set 8 on which sterilization processing has not been performed in some cases. Moreover, conventionally, the operator has to know the term of validity of sterilization processing of the instrument set 8, and there is a probability that some operators erroneously store the instrument set 8 for which the term of validity of sterilization processing has not expired in some cases.

Thus, in this preferred embodiment, the storage management system 100 illustrated in FIG. 1 performs management of stocking of the instrument set 8 in the storage process 16 (see FIG. 2) and also performs determination of a wrong rack of the instrument set 8, determination of whether sterilization processing has been performed, and determination of a term of validation of sterilization processing. In a case of a wrong rack of the instrument set 8, if sterilization processing has not been performed or if a term of validity of sterilization processing has expired, notification is made before the instrument set 8 is actually stored.

In this preferred embodiment, the storage management system 100 includes a reading device 30, a display device 50, an operation device 55, and a storage management device 60.

The reading device 30 is operated by the operator and reads the target sterilization ID 22 by reading the sterilization identification mark 21 of the sterilization label 20 assigned to the instrument set 8. Furthermore, the reading device 30 can read the target rack ID 26 by reading the rack identification mark 25 assigned to the rack number 3 of the rack 2. Although not illustrated, the reading device 30 is provided with a handle that is gripped by the operator's hand.

Note that there is no particular limitation on a type of the reading device 30, and the reading device 30 may be a device that can read the target sterilization ID 22 of the sterilization identification mark 21 and the target rack ID 26 of the rack identification mark 25. For example, in a case where the sterilization identification mark 21 and the rack identification mark 25 are two-dimensional codes, the reading device 30 may be a two-dimensional code reader that can read two-dimensional codes. For example, in a case where the sterilization identification mark 21 and the rack identification mark 25 are IC tags, the reading device 30 may be an IC tag reader. In this preferred embodiment, the reading device 30 is a non-contact type device. However, the reading device 30 may be a contact type device. Note that the number of the reading devices 30 used in the storage process 16 may be one and may be plural. An installation site of the reading device 30 is, for example, the storage area in which the rack 2 in which the instrument set 8 is stored in the storage process 16 is arranged.

A screen that is used in stocking the instrument set 8 in the storage process 16 is displayed on the display device 50. Note that there is no particular limitation on a type of the display device 50. For example, the display device 50 may be a display of a mobile terminal. The display device 50 may be a display of a desktop type or notebook type (in other words, laptop type) personal computer.

The operation device 55 is used when the operator operates a screen displayed on the display device 50 or the like. The screen displayed on the steering 50 can be switched, for example, by the operator's operation of the operation device 55. Moreover, in which rack number 3 of which rack 2 the instrument set 8 is to be stocked can be registered in the storage management device 60 by the operator's operation of the operation device 55. Note that there is no particular limitation on a type of the operation device 55. For example, the operation device 55 is, for example, a keyboard, a mouse, or the like of the personal computer. However, the operation device 55 may be a touch panel provided on the display device 50. Note that each of the number of the display devices 50 and the number of the operation devices 55 is one herein, but may be plural.

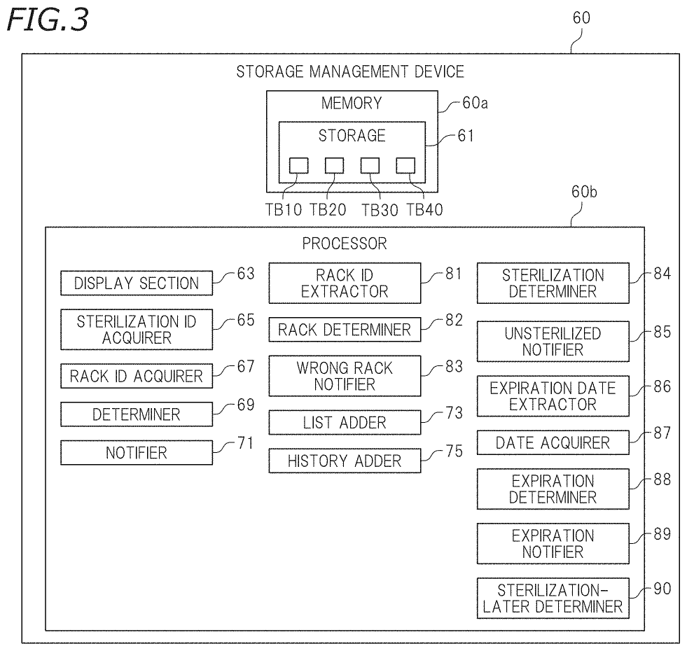

Next, the storage management device 60 according to this preferred embodiment will be described. The storage management device 60 manages storage of the instrument set 8 in the storage process 16 (see FIG. 2), that is, a stock history. Thus, when the instrument set 8 was stocked in the rack number 3 of the predetermined rack 2 can be managed. The storage management device 60 also notifies a wrong rack of the instrument set 8 and expiration of a term of validity of sterilization processing. The storage management device 60 is a computer control device. FIG. 3 is a block diagram of the storage management device 60. As illustrated in FIG. 3, the storage management device 60 includes a memory 60a and at least one processor 60b. The number of the processors 60b may be one and may be plural. For example, the processor 60b uses a program stored in the memory 60a, and thus, can execute processing. The storage management device 60 may be realized by a dedicated computer and may be realized by a general-purpose computer.

In this preferred embodiment, as illustrated in FIG. 1, the storage management device 60 is communicably connected to the reading device 30. The storage management device 60 may be connected to the reading device 30 via a wired communication and may be connected thereto via a wireless communication. The storage management device 60 is communicably connected to the display device 50 and the operation device 55. Note that the storage management device 60, the display device 50, and the operation device 55 may be realized by one personal computer.

Herein, the storage management system 100 may be realized by a so-called client-server system, and may be realized by a cloud computing. Moreover, the storage management system 100 may be realized by a so-called stand-alone system.

In this preferred embodiment, as illustrated in FIG. 3, the storage management device 60 includes a storage 61, a display section 63, a sterilization ID acquirer 65, a rack ID acquirer 67, a determiner 69, a notifier 71, a list adder 73, and a history adder 75. The storage management device 60 further includes a rack ID extractor 81, a rack determiner 82, a wrong rack notifier 83, a sterilization determiner 84, an unsterilized notifier 85, an expiration date extractor 86, a date acquirer 87, an expiration determiner 88, an expiration notifier 89, and a sterilization-later determiner 90.

The storage 61 is stored in the memory 60a. The display section 63, the sterilization ID acquirer 65, the rack ID acquirer 67, the determiner 69, the notifier 71, the list adder 73, the history adder 75, the rack ID extractor 81, the rack determiner 82, the wrong rack notifier 83, the sterilization determiner 84, the unsterilized notifier 85, the expiration date extractor 86, the date acquirer 87, the expiration determiner 88, the expiration notifier 89, and the sterilization-later determiner 90 can be executed by the processor 60b.

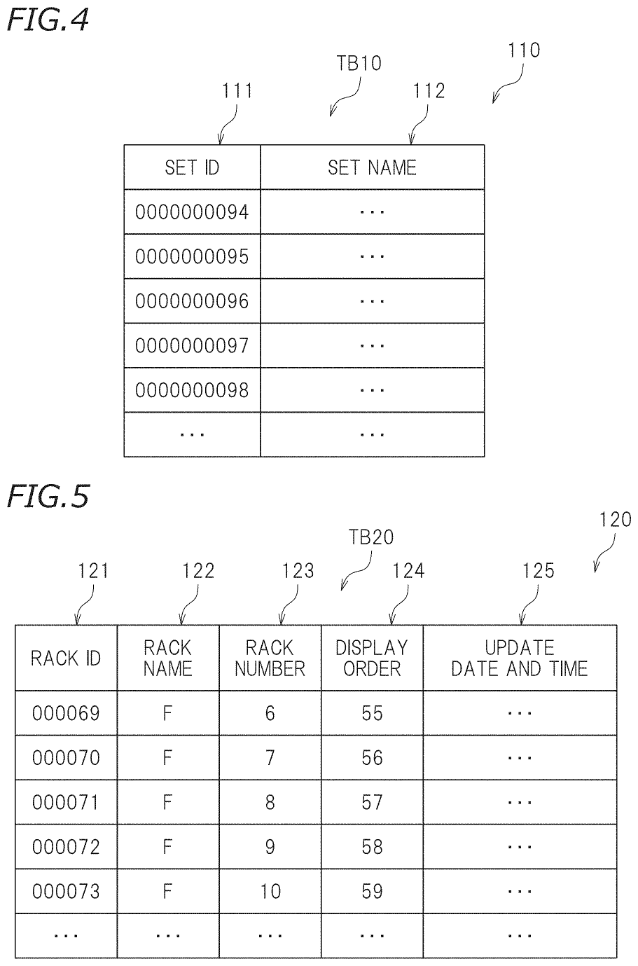

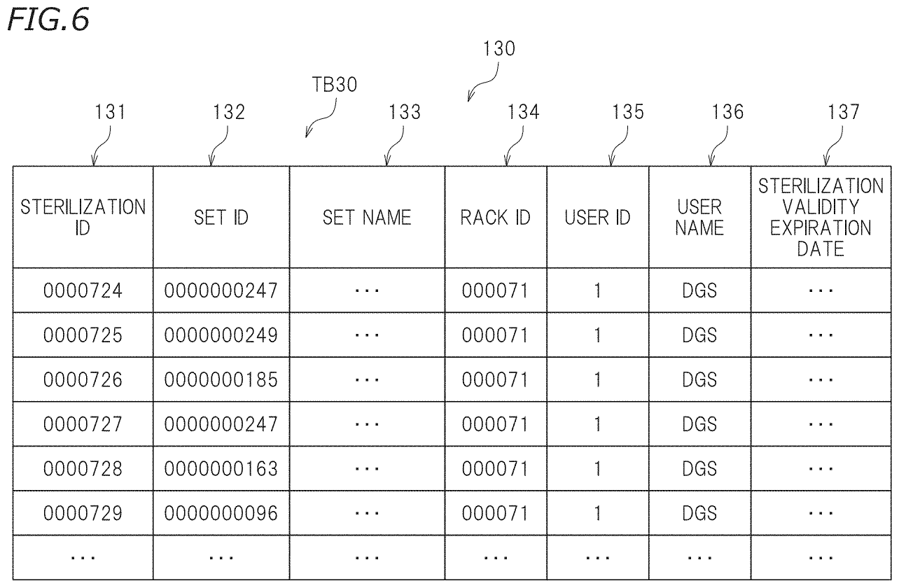

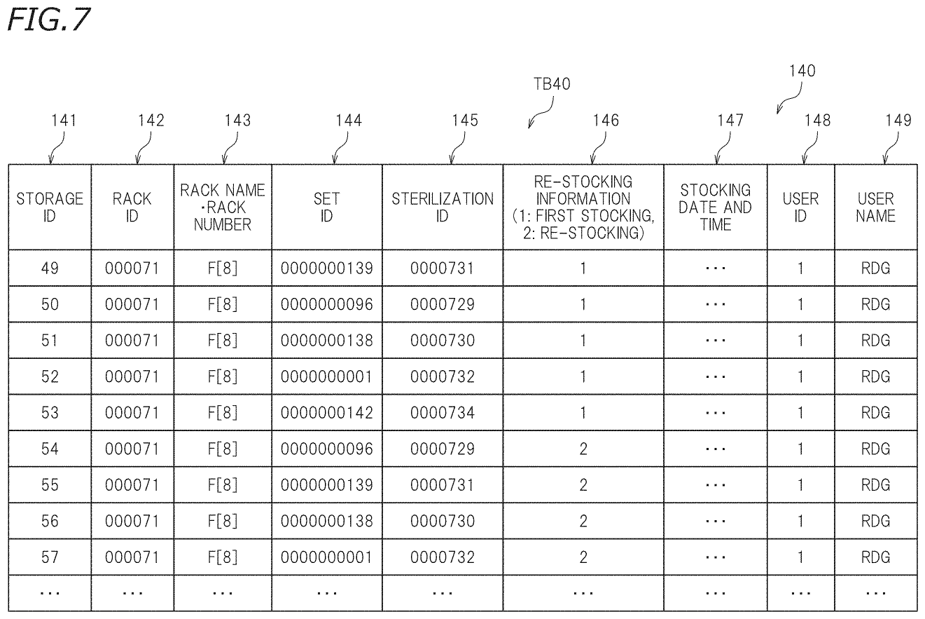

In this preferred embodiment, in the storage 61 realized by the memory 60a, an instrument set master table TB10 (see FIG. 4), a rack master table TB20 (see FIG. 5), a sterilization table TB30 (see FIG. 6), a storage history table TB40 (see FIG. 7) are stored in advance.

As illustrated in FIG. 4, the instrument set master table TB10 is a table in which instrument set information 110 is stored. The instrument set information 110 is, for example, information in which at least a set ID 111 and a set name 112 are associated with one another. In other words, in the instrument set information 110, there are at least items of the set ID 111, the set name 112, and the like.

As used herein, “associated with one another” represents a state where items are described in the same row of the table. “Associated with one another” can be paraphrased as “corresponding to one another.”

As illustrated in FIG. 4, the set ID 111 is a specific ID assigned to each instrument set 8. The set ID 111 is a character string including a number, an alphabet, or the like, and differs for each instrument set 8. The set name 112 is a name of the instrument set 8. The set name 112 may be arbitrarily given to the instrument set 8, for example, by the operator or the like. In this preferred embodiment, the set name 112 is uniquely determined from the set ID 111.

As illustrated in FIG. 5, the rack master table TB20 is a table in which rack information 120 is stored. The rack information 120 is information in which, for example, at least a rack ID 121, a rack name 122, a rack number 123, a display order 124, and an update date and time 125 are associated with one another. Herein, in the rack information 120, there are at least items of the rack ID 121, the rack name 122, the rack number 123, the display order 124, the update date and time 125, and the like.

The rack ID 121 is a specific ID assigned to each rack number 3 of each rack 2 (see FIG. 1) in which the instrument set 8 is stored. The rack ID 121 is a character string including a number, an alphabet, or the like, and differs for each rack number 3 of each rack 2. The rack name 122 is a name of the rack 2. The rack name 122 is arbitrarily given to the rack 2, for example, by the operator or the like. The rack number 123 is a rack number assigned to each rack 2, and is the rack number 3 of the rack 2 (see FIG. 1). In this preferred embodiment, the rack number 3 of the rack 2 indicated in FIG. 1 or the rack number 3a of the sterilization-later rack 2a is specified from the rack ID 121.

The display order 124 is an order in displaying a list of the rack numbers 3 of the racks 2 illustrated in FIG. 1. For example, the list of the rack numbers 3 of the racks 2 is displayed in an ascending order or a descending order of the display order 124. The update date and time 125 is date and time on which the rack information 120 was updated. Herein, when any one of the items of the rack ID 121, the rack name 122, the rack number 123, and the display order 124 associated with the update date and time 125 is changed, the time and date on which the item is changed is added to the update date and time 125.

As illustrated in FIG. 6, the sterilization table TB30 is a table in which sterilization information 130 for the instrument set 8 is stored. The sterilization information 130 is information in which, for example, at least a sterilization ID 131, a set ID 132, a set name 133, a rack ID 134, a user ID 135, a user name 136, and a sterilization validity term expiration date 137 are associated with one another. Herein, in the sterilization information 130, there are at least items of the sterilization ID 131, the set ID 132, the set name 133, the rack ID 134, the user ID 135, the user name 136, the sterilization validity term expiration date 137, and the like. In this preferred embodiment, the sterilization information 130 is an example of storage destination information and sterilization information according to a preferred embodiment of the present invention, and is information achieved by combining the storage destination information and the sterilization information according to a preferred embodiment of the present invention together.

The sterilization ID 131 is a specific ID assigned to the instrument set 8 on which sterilization processing has been performed in the sterilization process 15 illustrated in FIG. 2. Herein, the instrument set 8 assigned the sterilization ID 131 is determined to have been sterilized. The instrument set 8 can be identified from the sterilization ID 131. The sterilization ID 131 is an example of an identification ID according to a preferred embodiment of the present invention. The set ID 132 is a set ID assigned to the instrument set 8 assigned the sterilization ID 131. The set ID 132 corresponds to the set ID 111 of the instrument set information 110 (see FIG. 4). The set name 133 illustrated in FIG. 6 is a set name of the instrument set 8 assigned the sterilization ID 131. The set name 133 corresponds to the set name 112 of the instrument set information 110 (see FIG. 4). Note that one of the set ID 132 and the set name 133 may be omitted.

The rack ID 134 illustrated in FIG. 6 is an ID assigned to the rack number 3 of the rack 2 in which the instrument set 8 assigned the sterilization ID 131 is stored. The rack ID 134 corresponds to the rack ID 121 of the rack information 120 (see FIG. 5). The user ID 135 illustrated in FIG. 6 is an ID of the operator who performed sterilization processing on the instrument set 8 assigned the sterilization ID 131. The user name 136 is a name of the operator who performed sterilization processing on the instrument set 8 assigned the sterilization ID 131. Herein, the user ID 135 and the user name 136 correspond to user information (not illustrated) stored in a user master table (not illustrated). The user name 136 can be uniquely determined from the user ID 135. Therefore, one of the user ID 135 and the user name 136 may be omitted.

The sterilization validity term expiration date 137 is an expiration date of a term of validity of sterilization processing that has been performed on the instrument set 8 assigned to the sterilization ID 131. The sterilization validity term expiration date 137 is set to be a date after a predetermined period from issuance of the sterilization label 20 (see FIG. 1). The predetermined period is set based on a type of sterilization processing, a type or types of one or more medical instruments 5 forming the instrument set 8 on which sterilization processing has been performed, or the like.

As illustrated in FIG. 7, the storage history table TB40 is a table in which storage history information 140 that is history information of the instrument set 8 that has been stored in the storage process 16 illustrated in FIG. 2 is stored. The storage history information 140 is information in which, for example, at least a storage ID 141, a rack ID 142, rack name and rack number 143, a set ID 144, a sterilization ID 145, re-stocking information 146, stocking date and time 147, a user ID 148, and a user name 149 are associated with one another. In the storage history information 140, there are at least items of the storage ID 141, the rack ID 142, the rack name and rack number 143, the set ID 144, the sterilization ID 145, the re-stocking information 146, the stocking date and time 147, the user ID 148, the user name 149, and the like.

The storage ID 141 is a specific ID assigned to the stored instrument set 8 when the instrument set 8 is stored in the storage process 16. The storage ID 141 is a character string including a number, an alphabet, or the like, and is issued each time the instrument set 8 is stocked. Therefore, each time one instrument set 8 circulates in the circulation cycle 10 and then is stored in the storage process 16, a different storage ID 141 is issued. Accordingly, a plurality of storage IDs 141 can be assigned to the one instrument set 8.

The rack ID 142 is a rack ID of the rack number 3 of the rack 2 (see FIG. 1) in which the instrument set 8 assigned the storage ID 141 is stored. The rack ID 142 corresponds to the rack ID 121 of the rack information 120 (see FIG. 5). The rack name and rack number 143 illustrated in FIG. 7 is a rack name and a rack number of a rack in which the instrument set 8 assigned the storage ID 141 is stored. Herein, the rack name and rack number 143 are indicated as “RACK NAME [RACK NUMBER].” The rack name and rack number 143 correspond to the rack name 122 (see FIG. 5) and the rack number 123 (see FIG. 5) of the rack information 120. The set ID 144 illustrated in FIG. 7 is a set ID assigned to the instrument set 8 assigned the storage ID 141. The set ID 144 corresponds to the set ID 111 of the instrument set information 110 (see FIG. 4). The sterilization ID 145 illustrated in FIG. 7 is a sterilization ID that is assigned when sterilization processing is performed on the instrument set 8 assigned the storage ID 141. The sterilization ID 145 corresponds to the sterilization ID 131 of the sterilization information 130 (see FIG. 6).

The re-stocking information 146 illustrated in FIG. 7 is information on whether the instrument set 8 assigned the storage ID 141 has been re-stocked. Re-stocking includes stocking the instrument set 8 that has not been used in the operation and examination process 11 again without performing sterilization processing in the sterilization process 15 on the instrument set 8 in the storage process 16. For example, if the associated instrument set 8 is not re-stocked but is stocked for the first time (herein, if stocking of the instrument set 8 is first stocking), “1” is set in the re-stocking information 146. On the other hand, if the associated instrument set 8 has been re-stocked, that is, stocking of the instrument set 8 is a second or subsequent stocking, “2” is set in the re-stocking information 146. However, a specific numerical value or character of the re-stocking information 146 can be set for each medical site as appropriate, the first stocking is not limited to “1,” and the re-stocking is not limited to “2.”

The stocking date and time 147 is a date and a time on which the instrument set 8 assigned the storage ID 141 was stocked, that is, the date and the time on which the instrument set 8 was stored in the rack number 3 of the rack 2. The user ID 148 is an ID of the operator who stocked the instrument set 8 assigned the storage ID 141. The user name 136 is a name of the operator who stocked the instrument set 8 assigned the storage ID 141. Herein, the user ID 148 and the user name 149 correspond to user information (not illustrated) stored in the above-described user master table (not illustrated). The user name 149 can be uniquely determined from the user ID 148, and therefore, one of the user ID 148 and the user name 149 may be omitted.

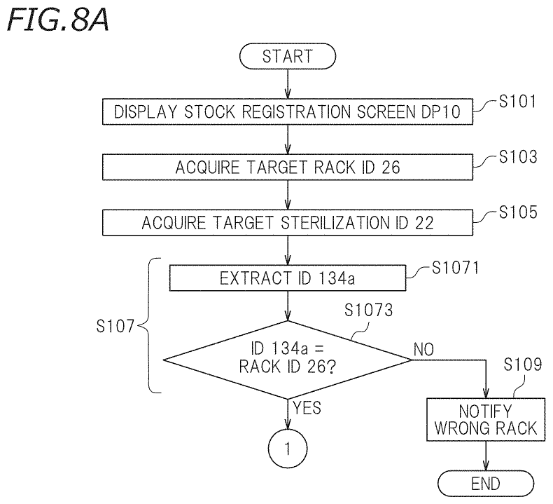

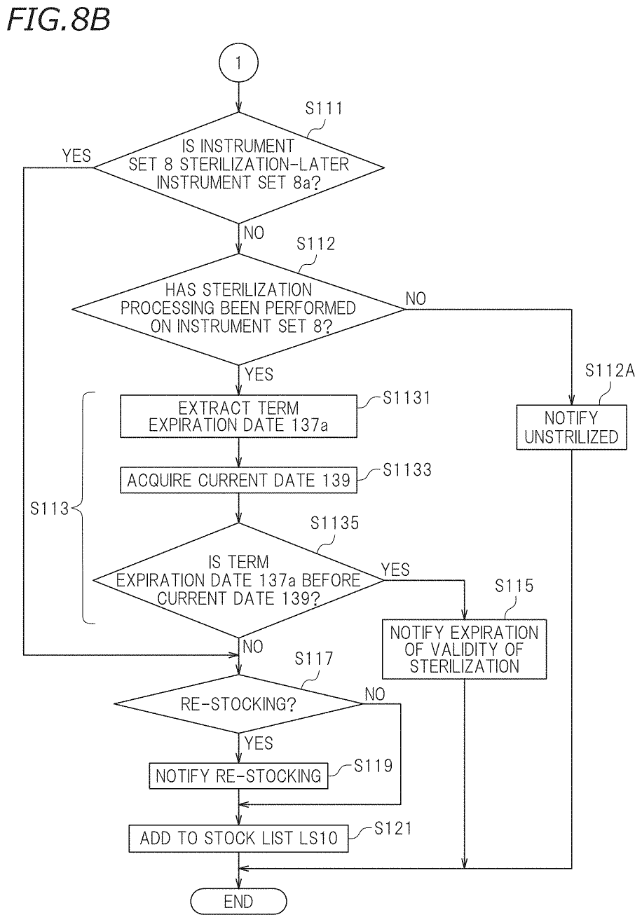

Next, process steps of processing of the storage management device 60 in stocking the instrument set 8 in the storage process 16 of FIG. 2 will be described with reference to flowcharts of FIG. 8A and FIG. 8B.

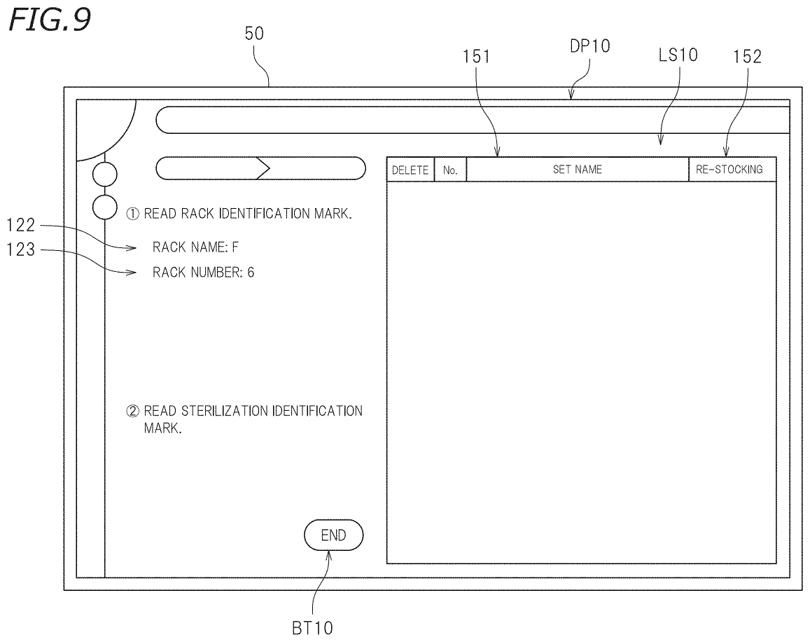

In this preferred embodiment, first, in Step S101 of FIG. 8A, in order to register one or more instrument sets 8 that are to be stocked in a rack number 3 of a predetermined rack 2 (see FIG. 1), the display section 63 of FIG. 3 displays a stock registration screen DP10 on the display device 50, as illustrated in FIG. 9. The stock registration screen DP10 is a screen used in stocking and storing the instrument set 8 in the rack number 3 of the predetermined rack 2 in the storage process 16 and also serves as a screen via which a stocking destination of the instrument set 8 is registered for the storage management device 60.

After the stock registration screen DP10 is displayed on the display device 50 in the above-described manner, in Step S103 of FIG. 8A, the rack ID acquirer 67 of FIG. 3 acquires the target rack ID 26 (see FIG. 1) assigned to the rack number 3 of the rack 2 that is the stocking destination of the instrument set 8. Herein, as illustrated in FIG. 1, the target rack ID 26 is read by the reading device 30. The operator holds a reader (not illustrated) of the reading device 30 over the rack identification mark 25 assigned to the rack number 3 of the rack 2 that is a target of stocking in stocking the instrument set 8. Thus, the target rack ID 26 recorded in the rack identification mark 25 can be read by the reading device 30. Accordingly, the rack ID acquirer 67 can acquire the target rack ID 26. Note that the target rack ID 26 acquired by the rack ID acquirer 67 is stored in the storage 61.

In this preferred embodiment, after the target rack ID 26 is acquired, the rack ID 121 that matches the target rack ID 26 is extracted from the rack master table TB20 of FIG. 5 and the rack name 122 and the rack number 123 associated with the extracted rack ID 121 are extracted. Then, as illustrated in FIG. 9, the display section 63 displays the rack name 122 and the rack number 123 that have been extracted on the stock registration screen DP10.

Next, in Step S105 of FIG. 8A, the operator registers the instrument set 8 that is to be stocked in the rack number 3 of the rack 2 assigned the target rack ID 26. Herein, the sterilization ID acquirer 65 of FIG. 3 first acquires the target sterilization ID 22 (see FIG. 1) of the instrument set 8 that is a target to be stocked. As illustrated in FIG. 1, the target sterilization ID 22 is read by the reading device 30. The operator holds the reader of the reading device 30 over the sterilization identification mark 21 of the sterilization label 20 attached to the instrument set 8 that is the target to be stocked. Thus, the target sterilization ID 22 recorded in the sterilization identification mark 21 is read by the reading device 30. Accordingly, the sterilization ID acquirer 65 can acquire the target sterilization ID 22. Note that the target sterilization ID 22 acquired by the sterilization ID acquirer 65 is stored in the storage 61.

After the target sterilization ID 22 is acquired by the sterilization ID acquirer 65 in the above-described manner, in Step S107 of FIG. 8A, whether the stocking destination of the instrument set 8 assigned the target sterilization ID 22 is proper is determined. In this preferred embodiment, a stocking destination indicating which rack number 3 of which rack 2 the instrument set 8 is to be stored is set for each instrument set 8 in advance. Herein, as illustrated in FIG. 6, the rack ID 134 of the sterilization information 130 of the sterilization table TB30 is an ID of the rack number 3 of the rack 2 of a proper stocking destination of the instrument set 8 assigned the sterilization ID 131.

In this preferred embodiment, determination of whether the stocking destination of the instrument set 8 is proper is performed by performing Step S1071 and Step S1073 in this order. First, in Step S1071, first, the rack ID extractor 81 of FIG. 3 extracts the rack ID 134 associated with the sterilization ID 131 that matches the target sterilization ID 22 from the sterilization information 130 of the sterilization table TB30 illustrated in FIG. 6 and determines the extracted rack ID 134 as an extracted rack ID 134a.

Next, in Step S1073 of FIG. 8A, the rack determiner 82 of FIG. 3 determines whether the extracted rack ID 134a matches the target rack ID 26 read by the reading device 30 and acquired by the rack ID acquirer 67 in Step S103. If the extracted rack ID 134a matches the target rack ID 26, it is determined that the stocking destination of the instrument set 8 assigned the target sterilization ID 22 is proper. In this case, the process proceeds to Step S111 next.

On the other hand, in Step S1073, if it is determined by the rack determiner 82 that the extracted rack ID 134a does not match the target rack ID 26, it is determined that the stocking destination of the instrument set 8 assigned the target sterilization ID 22 is improper and a wrong rack is determined. In this case, the process proceeds to Step S109 next. In Step S109, the wrong rack notifier 83 of FIG. 3 notifies the operator of a wrong rack. There is no particular limitation on a method for notifying the wrong rack.

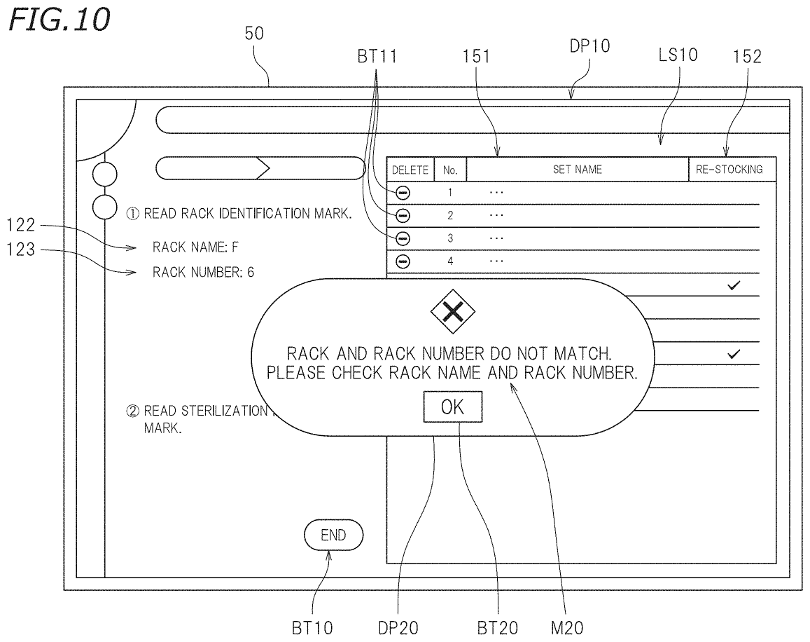

In this preferred embodiment, as illustrated in FIG. 10, the wrong rack notifier 83 displays a wrong rack warning screen DP20 via which a wrong rack is notified on the display device 50. The wrong rack warning screen DP20 is displayed, for example, so as to be superimposed on the stock registration screen DP10. On the wrong rack warning screen DP20, a wrong rack notification message M20 saying, “RACK AND RACK NUMBER DO NOT MATCH. PLEASE CHECK RACK NAME AND RACK NUMBER.” or the like is displayed and also an OK button BT20 is displayed. The operator can know that the rack number 3 of the rack 2 of the stocking destination of the instrument set 8 assigned the target sterilization ID 22 read by the reading device 30 is not proper by viewing the wrong rack notification message M20. Moreover, the operator presses the OK button BT20 via the operation device 55 to close the wrong rack warning screen DP20. Herein, as an example of the method for notifying a wrong rack, the wrong rack warning screen DP20 is displayed. However, the wrong rack notifier 83 may be configured to notify the operator of a wrong rack, for example, by causing an unillustrated speaker to make a warning sound, such as a buzzer sound or the like.

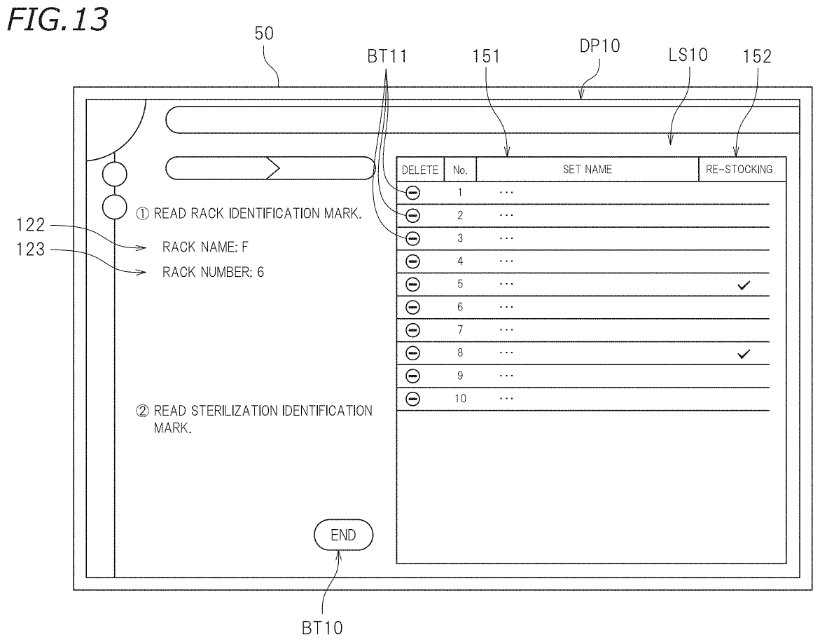

Note that, in this preferred embodiment, in Step S107 of FIG. 8A, when a wrong rack is determined, the instrument set 8 assigned the target sterilization ID 22 is not added to a stock list LS10 (see FIG. 13) that will be described later and a flow of process steps of the flowcharts of FIG. 8A and FIG. 8B is terminated.

In this preferred embodiment, in Step S111 of FIG. 8B, the sterilization-later determiner 90 of FIG. 3 determines whether the instrument set 8 assigned the target sterilization ID 22 is the sterilization-later instrument set 8a. As described above, for the sterilization-later instrument set 8a, it is set in advance that the sterilization-later instrument set 8a is stored in the rack number 3a of the sterilization-later rack 2a. Information related to the sterilization-later rack ID 26a (see FIG. 1) assigned to the rack number 3a of the sterilization-later rack 2a is stored in the storage 61 of the memory 60a in advance. Thus, in this preferred embodiment, the sterilization-later determiner 90 determines whether the rack ID 134 (see FIG. 6) of the sterilization information 130 corresponding to the target sterilization ID 22 is the sterilization-later rack ID 26a. Herein, the rack ID 134 corresponding to the target sterilization ID 22 is the extracted rack ID 134a extracted by the rack ID extractor 81 in Step S1071 described above. Therefore, the sterilization-later determiner 90 determines whether the extracted rack ID 134a matches the sterilization-later rack ID 26a.

If it is determined by the sterilization-later determiner 90 that the extracted rack ID 134a matches the sterilization-later rack ID 26a, it is determined that the stocking destination of the instrument set 8 assigned the target sterilization ID 22 is the rack number 3a of the sterilization-later rack 2a (see FIG. 1). In this case, it is determined that the instrument set 8 assigned the target sterilization ID 22 is the sterilization-later instrument set 8a. As described above, in a case where the instrument set 8 assigned the target sterilization ID 22 is the sterilization-later instrument set 8a, the instrument set 8 that is a target to be stocked is the sterilization-later instrument set 8a that is sterilized after being stored, and therefore, the process proceeds to Step S117 of FIG. 8B next without performing determination of whether sterilization processing has been performed in Step S112 in FIG. 8B and determination of a term of validity of sterilization processing in Step S113. Note that, in a case where it is determined that the instrument set 8 assigned the target sterilization ID 22 is the sterilization-later instrument set 8a, a sterilization-later instrument set notification screen (not illustrated) including a message indicating that the instrument set 8 is the sterilization-later instrument set 8a is displayed on the display device 50.

On the other hand, in Step S111, if it is determined by the sterilization-later determiner 90 that the extracted rack ID 134a does not match the sterilization-later rack ID 26a, it is determined that the stocking destination of the instrument set 8 assigned the target sterilization ID 22 is not the rack number 3a of the sterilization-later rack 2a (see FIG. 1). In this case, it is determined that the instrument set 8 assigned the target sterilization ID 22 is not the sterilization-later instrument set 8a. As described above, in a case where the instrument set 8 assigned the target sterilization ID 22 is not the sterilization-later instrument set 8a, sterilization processing has been performed before the instrument set 8 is stored, and therefore, the process proceeds to Step S112 next.

In Step S112, whether sterilization processing has been performed on the instrument set 8 assigned the target sterilization ID 22 is determined. In this preferred embodiment, if sterilization processing has been performed on the instrument set 8, there is the sterilization ID 131 that matches the target sterilization ID 22 in the sterilization information 130 of the sterilization label information table TB30. Thus, the sterilization determiner 84 of FIG. 3 determines whether there is the sterilization ID 131 that matches the target sterilization ID 22 in the sterilization information 130.

If it is determined that there is not the sterilization ID 131 that matches the target sterilization ID 22 in the sterilization information 130, it is determined that sterilization processing has not been performed on the instrument set 8 assigned the target sterilization ID 22. In this case, the process proceeds to Step S112A next. In Step S112A, the unsterilized notifier 85 of FIG. 3 notifies the operator that the instrument set 8 is unsterilized (that sterilization processing has not been performed on the instrument set 8). There is no particular limitation on a method for notifying that the instrument set 8 is unsterilized.

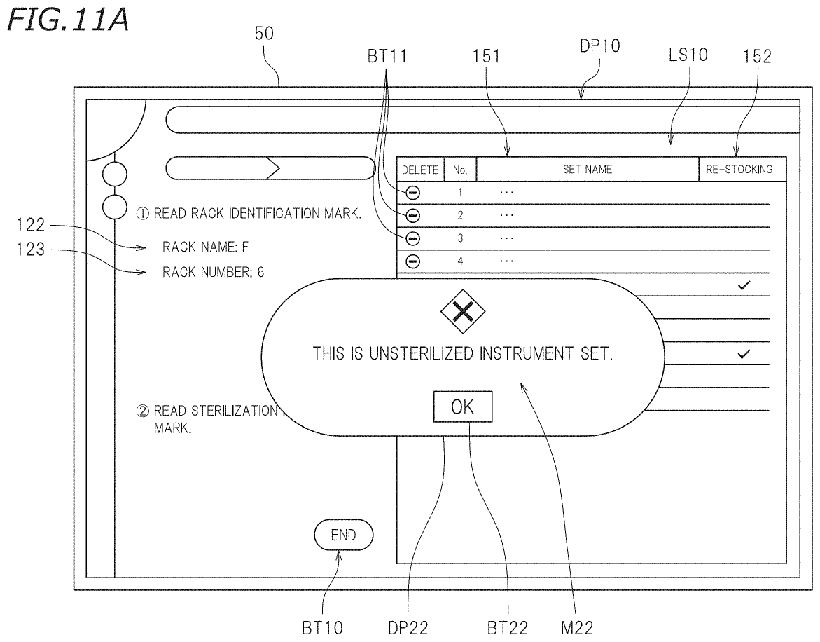

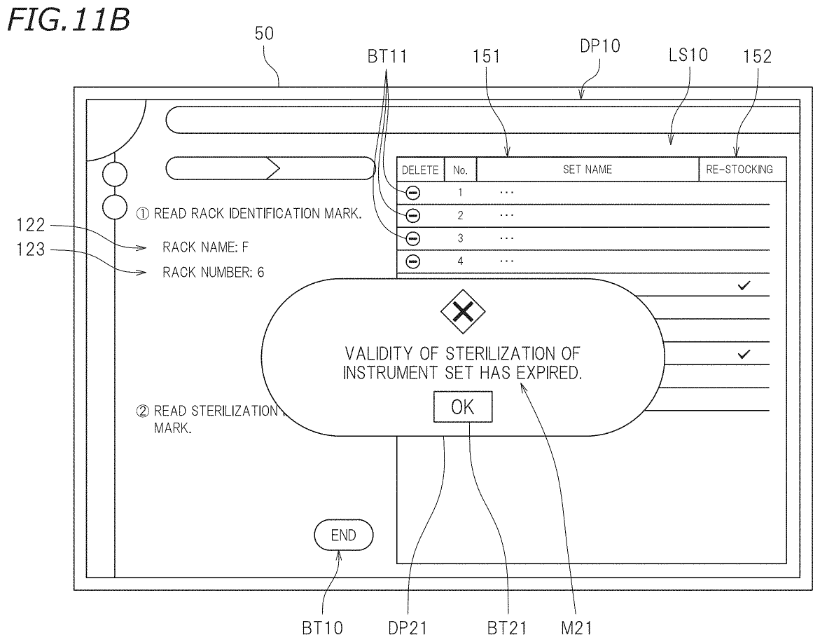

In this preferred embodiment, as illustrated in FIG. 11A, the unsterilized notifier 85 displays an unsterilized warning screen DP22 that notifies that the instrument set 8 is unsterilized on the display device 50. The unsterilized warning screen DP22 is displayed, for example, so as to be interposed on the stock registration screen DP10. On the unsterilized warning screen DP22, an unsterilized notification message M22 saying, “THIS IS UNSTERILIZED INSTRUMENT SET.” or the like is displayed and also an OK button BT22 is displayed. The operator can know that sterilization processing has not been performed on the instrument set 8 assigned the target sterilization ID 22 read by the reading device 30 by viewing the unsterilized notification message M22. The operator presses the OK button BT22 via the operation device 55 to close the unsterilized warning screen DP22. Herein, as an example of a method for notifying that the instrument set 8 is unsterilized, the unsterilized warning screen DP22 is displayed. However, the unsterilized notifier 85 may be configured to notify that the instrument set 8 is unsterilized, for example, by causing an unillustrated speaker to make a warning sound, such as a buzzer sound or the like.

On the other hand, in Step S112, if it is determined that there is the sterilization ID 131 that matches the target sterilization ID 22 in the sterilization information 130, it is determined that sterilization processing has been performed on the instrument set 8 assigned the target sterilization ID 22. In this case, the process proceeds to Step S113 next.

In Step S113, whether sterilization processing that has been performed on the instrument set 8 assigned the target sterilization ID 22 is still in a valid period is determined. In this preferred embodiment, as described above, when sterilization processing is performed on the instrument set 8 and the sterilization label 20 (see FIG. 1) is issued, a term of validity of the sterilization processing of the instrument set 8 is set. The term of validity of sterilization processing is set based on a type of sterilization processing or a type or types of one or more medical instruments 5 forming the instrument set 8. In this preferred embodiment, as illustrated in FIG. 6, as the sterilization validity term expiration date 137 of the sterilization information 130 of the sterilization table TB30, a term of validity of sterilization processing for the instrument set 8 assigned the sterilization ID 131 is set.

In this preferred embodiment, determination of whether the term of validity of sterilization processing has expired is performed by performing Step S1131, Step S1133, and Step S1135 in this order. First, in Step S1131, the expiration date extractor 86 of FIG. 3 extracts the sterilization validity term expiration date 137 associated with the sterilization ID 131 that matches the target sterilization ID 22 from the sterilization table TB30 of FIG. 6 and determines the extracted sterilization validity term expiration date 137 as an extracted sterilization validity term expiration date 137a. Next, in Step S1133, the date acquirer 87 of FIG. 3 acquires a current date 139 that is a date of a present day. In this preferred embodiment, in the storage management device 60, date and time information related to current date and time is set. The date acquirer 87 acquires the current date 139, based on the current date and time information.

Next, in Step S1135, the expiration determiner 88 of FIG. 3 determines whether the extracted sterilization validity term expiration date 137a is before the current date 139. Herein, if it is determined by the expiration determiner 88 that the extracted sterilization validity term expiration date 137a is the same as the current date 139 or after the current date 139, it is determined that a term of validity of sterilization processing for the instrument set 8 assigned the target sterilization ID 22 has not expired. In this case, the process proceeds to Step S117 of FIG. 8B next.

On the other hand, in Step S1135 of FIG. 8B, if it is determined by the expiration determiner 88 that the extracted sterilization validity term expiration date 137a is before the current date, it is determined that the term of validity of the sterilization processing for the instrument set 8 assigned the target sterilization ID 22 has expired, and thus, expiration of validity of sterilization is determined. In this case, the process proceeds to Step S115 next. In Step S115, the expiration notifier 89 of FIG. 3 notifies the operator of expiration of validity of sterilization. There is no particular limitation on a method for notifying expiration of validity of sterilization.

In this preferred embodiment, as illustrated in FIG. 11B, the expiration notifier 89 displays a sterilization validity expiration warning screen DP 21 that notifies that validity of sterilization has expired on the display device 50. The sterilization validity expiration warning screen DP 21 is displayed, for example, so as to be superimposed on the stock registration screen DP10. On the sterilization validity expiration warning screen DP 21, a sterilization validity expiration notification message M21 saying, “VALIDITY OF STERILIZATION OF INSTRUMENT SET HAS EXPIRED.” or the like is displayed and also an OK button BT21 is displayed. The operator can know that the term of validity of the sterilization processing for the instrument set 8 assigned the target sterilization ID 22 read by the reading device 30 has expired by viewing the sterilization validity expiration notification message M21. Moreover, the operator presses the OK button BT21 via the operation device 55 to close the sterilization validity expiration warning screen DP 21. Herein, as an example of a method for notifying expiration of validity of sterilization, the sterilization validity expiration warning screen DP 21 is displayed. However, the expiration notifier 89 may be configured to notify the operator of expiration of validity of sterilization, for example, by causing an unillustrated speaker to make a warning sound, such as a buzzer sound or the like.

Note that, in this preferred embodiment, in Step S113 of FIG. 8B, when expiration of validity of sterilization is determined, the instrument set 8 assigned the target sterilization ID 22 is not added to the stock list LS10 and the flow of process steps of the flowcharts of FIG. 8A and FIG. 8B is terminated. If expiration of validity of sterilization is determined, the process returns to the sterilization process 15 of FIG. 2, sterilization processing is performed on the instrument set 8, and a new sterilization label 20 is issued. Then, after the new sterilization label 20 is issued, the storage process 16 is performed again.

In this preferred embodiment, after the target sterilization ID 22 is acquired by the sterilization ID acquirer 65, in Step S117 of FIG. 8B, the determiner 69 of FIG. 3 determines whether the instrument set 8 assigned the target sterilization ID 22 has been re-stocked. In order to determine whether the instrument set 8 has been re-stocked, the determiner 69 determines whether there is the sterilization ID 145 that matches the target sterilization ID 22 in the storage history information 140 (see FIG. 7). Herein, the determiner 69 determines whether there is an ID that matches the target sterilization ID 22 among the sterilization IDs 145 of the storage history information 140 stored in the storage history table TB40 illustrated in FIG. 7.

In this preferred embodiment, if the sterilization ID 145 that matches the target sterilization ID 22 is included in the storage history information 140, after sterilization processing that is to be performed when the sterilization label 20 (see FIG. 1) of the sterilization identification mark 21 in which the target sterilization ID 22 is recorded is issued is performed, the instrument set 8 assigned the target sterilization ID 22 has been already stocked at least once. That is, although the instrument set 8 assigned the target sterilization ID 22 has been dispensed once, the instrument set 8 has not been used in the operation and examination process 11 and has been re-stocked without going through the sterilization processing. As described above, if the sterilization ID 145 that matches the target sterilization ID 22 is included in the storage history information 140, the determiner 69 determines that the instrument set 8 assigned the target sterilization ID 22 has been re-stocked. If it is determined that the instrument set 8 assigned the target sterilization ID 22 has been re-stocked in the above-described manner, the process proceeds to Step S119 of FIG. 8B next.

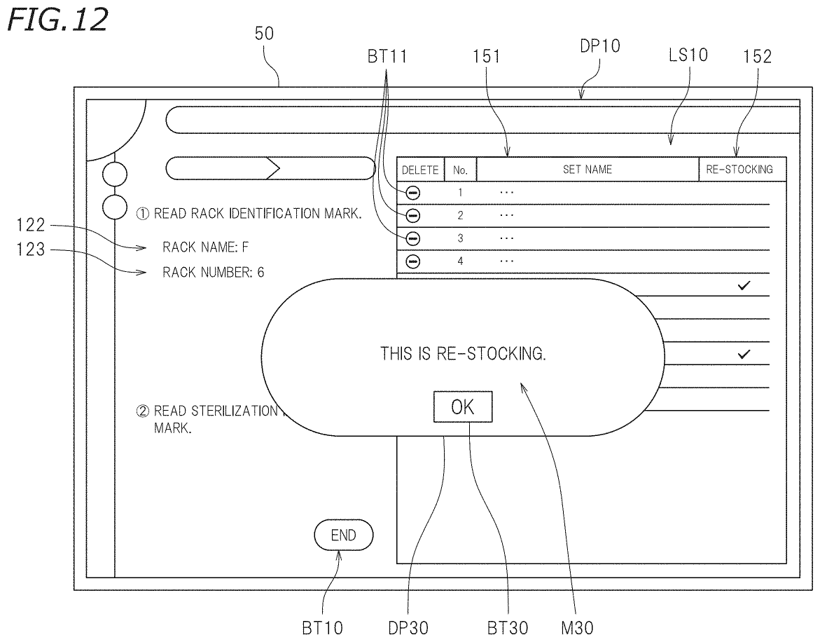

In Step S119, the notifier 71 of FIG. 3 notifies that the instrument set 8 assigned the target sterilization ID 22 has been re-stocked. There is no particular limitation on a method for notifying re-stocking. In this preferred embodiment, as illustrated in FIG. 12, the notifier 71 displays a re-stocking warning screen DP30 that notifies re-stocking on the display device 50. The re-stocking warning screen DP30 is displayed, for example, so as to be superimposed on the stock registration screen DP10. On the re-stocking warning screen DP30, a re-stocking notification message M30 saying, “THIS IS RE-STOCKING.” or the like is displayed and also an OK button BT30 is displayed. The operator can know that the instrument set 8 assigned the target sterilization ID 22 read by the reading device 30 has been re-stocked by viewing the re-stocking notification message M30. Moreover, the operator presses the OK button BT30 via the operation device 55 to close the re-stocking warning screen DP30. In this preferred embodiment, as illustrated in FIG. 8B, after Step S119, the process proceeds to Step S121.

On the other hand, in Step S117, if it is determined by the determiner 69 that the sterilization ID 145 that matches the target sterilization ID 22 is not included in the storage history information 140 (see FIG. 7), after sterilization processing when the sterilization label 20 (see FIG. 1) of the sterilization identification mark 21 in which the target sterilization ID 22 is recorded is issued is performed, the instrument set 8 assigned the target sterilization ID 22 is stocked for the first time (in other words, stocking of the instrument set 8 is first stocking). That is, after the instrument set 8 assigned the target sterilization ID 22 was used in the operation and examination process 11, sterilization processing has been performed on the instrument set 8 in the sterilization process 15. As described above, if the sterilization ID 145 that matches the target sterilization ID 22 is not included in the storage history information 140, the determiner 69 determines that the instrument set 8 assigned the target sterilization ID 22 is not re-stocked but is stocked for the first time.

Herein, if it is determined by the determiner 69 that the instrument set 8 is not re-stocked, the notifier 71 does not notify re-stocking of the instrument set 8 assigned the target sterilization ID 22. That is, the re-stocking warning screen DP30 illustrated in FIG. 12 is not displayed on the display device 50 and the process proceeds to Step S121 without performing processing of Step S119 of FIG. 8B.

In this preferred embodiment, in Step S121, the list adder 73 of FIG. 3 adds the instrument set 8 assigned the target sterilization ID 22 to the stock list LS10 illustrated in FIG. 13. Herein, whether or not the instrument set 8 assigned the target sterilization ID 22 has been re-stocked, the instrument set 8 is added to the stock list LS10. The stock list LS10 is a list displayed on the stock registration screen DP10 by the display section 63, and is a list of the instrument sets 8 that are to be stocked in the rack numbers 3 of the racks 2 assigned the target racks ID 26 acquired via the reading device 30.

The stock list LS10 includes items of a set name 151 and re-stocking information 152. The set name 151 is a set name of the instrument set 8 assigned the target sterilization ID 22. In this preferred embodiment, the sterilization ID 131 that matches the target sterilization ID 22 is extracted from the sterilization information 130 of the sterilization table TB30 illustrated in FIG. 6 and the set name 133 associated with the extracted sterilization ID 131 is extracted. The extracted set name 133 is a set name corresponding to the target sterilization ID 22. Herein, when the instrument set 8 assigned the target sterilization ID 22 is added to the stock list LS10, the list adder 73 adds the extracted set name 133 to the item of the set name 151 of the stock list LS10 displayed on the stock registration screen DP10 to display the extracted set name 133 thereon.

In this preferred embodiment, the re-stocking information 152 of the stock list LS10 is an item indicating whether the instrument set 8 added to the stock list LS10 has been re-stocked. For example, if the instrument set 8 added to the stock list LS10 has been re-stocked, the list adder 73 adds that the instrument set 8 has been re-stocked to the stock list LS10 by entering a check in the re-stocking information 152. In this preferred embodiment, if it is determined that there is the sterilization ID 145 that matches the target sterilization ID 22 in the storage history information 140 (see FIG. 7) and the instrument set 8 assigned the target sterilization ID 22 has been re-stocked, a check is entered in the re-stocking information 152. Note that processing of entering a check in the re-stocking information 152 may be performed by the notifier 71.

In this preferred embodiment, a delete button BT11 corresponding to each row of the stock list LS10 is provided in the stock list LS10. Herein, the operator presses the delete button BT11 via the operation device 55 to delete the instrument set 8 in a row corresponding to the delete button BT11 from the stock list LS10.

In this preferred embodiment, if a plurality of instrument sets 8 are stocked in the rack number 3 of the rack 2 assigned the target rack ID 26, the target sterilization IDs 22 assigned to the plurality of instrument sets 8 can be successively read by the reading device 30. In this case, each time one of the target sterilization IDs 22 assigned to the plurality of instrument sets 8 is read by the reading device 30, processes of Step S105 to Step S121 of FIG. 8A and FIG. 8B are sequentially executed. Each time one of the target sterilization IDs 22 is read by the reading device 30 and the target sterilization ID 22 is acquired by the sterilization ID acquirer 65, the instrument set 8 assigned the target sterilization ID 22 is added to the stock list LS10.

Herein, after all of the instrument sets 8 that are stocked in the rack number 3 of the rack 2 assigned the target rack ID 26 are added to the stock list LS10, as illustrated in FIG. 13, the operator presses a termination button BT10 displayed on the stock registration screen DP10 via the operation device 55. When the termination button BT10 is pressed, it is determined that the instrument sets 8 added to the stock list LS10 have been stocked in the rack number 3 of the rack 2 assigned the target rack ID 26. Herein, the storage history information 140 related to the instrument sets 8 added to the stock list LS10 is added to the storage history table TB40 illustrated in FIG. 7, so that it is confirmed that the instrument sets 8 have been stocked in the rack number 3 of the rack 2 assigned the target rack ID 26.

When the termination button BT10 is pressed, the history adder 75 of FIG. 3 adds the storage history information 140 related to each instrument set 8 added to the stock list LS10 to the storage history table TB40. Herein, the history adder 75 automatically issues a specific storage ID 141 to each instrument set 8 in the stock list LS10. The history adder 75 adds the target rack ID 26 to the rack ID 142. The history adder 75 extracts the rack name 122 and the rack number 123 associated with the target rack ID 26 from the rack information 120 of the rack master table TB20 (see FIG. 5) and adds the extracted rack name 122 and rack number 123 to the rack name and rack number 143. The history adder 75 extracts the set ID 132 associated with the target sterilization ID 22 from the sterilization information 130 of the sterilization table TB30 (see FIG. 6) and adds the extracted set ID 132 to the set ID 144 of the storage history information 140.

The history adder 75 adds the target sterilization ID 22 to the sterilization ID 145. Information on whether the instrument set 8 assigned the target sterilization ID 22 has been re-stocked is added to the re-stocking information 146. For example, in the stock list LS10, if a check has not been entered in any one of items of the re-stocking information 152, the history adder 75 determines first stocking and adds “1” to the re-stocking information 146. On the other hand, in the stock list LS10, if a check has been entered in any one of the items of the re-stocking information 152, the history adder 75 determines re-stocking, that is, in other words, second or subsequent stocking, and adds “2” to the re-stocking information 146.

The history adder 75 adds a date and a time on which the termination button BT10 was pressed to the stocking date and time 147. The history adder 75 adds the user ID and the user name of the operator who stocked the instrument set 8 to the user ID 148 and the user name 149, respectively. In this preferred embodiment, when the stock registration screen DP10 is displayed on the display device 50, the user ID of the operator is input. The user ID input by the operator at this time is added to the user ID 148 of the storage history information 140, and the user name corresponding to the user ID input by the operator is added to the user name 149 of the storage history information 140.

As described above, after the storage history information 140 related to the instrument set 8 is added to the stock list LS10 to the storage history table TB40 illustrated in FIG. 7, the operator stocks the instrument set 8 added to the stock list LS10 to the rack number 3 of the rack 2 assigned the target rack ID 26, that is, stores the instrument set 8 in the rack number 3 of the rack 2. In the above-described manner, stocking of the instrument set 8 in the storage process 16 is completed.

As described above, in this preferred embodiment, as illustrated in FIG. 3, the storage management device 60 for the medical instrument 5 includes the memory 60a and the at least one processor 60b. The memory 60a stores the sterilization information 130 (see FIG. 6) that is an example of storage destination information of a preferred embodiment of the present invention. The sterilization information 130 is information in which at least the sterilization ID 131 that is an example of the identification ID used to identify the instrument set 8 and the rack ID 134 assigned to the rack number 3 of the rack 2 in which the instrument set 8 is stored are associated with one another. The at least one processor 60b is configured or programmed to execute rack ID acquisition processing, instrument ID acquisition processing, rack ID extraction processing, rack determination processing, and wrong rack notification processing. The rack ID acquisition processing is executed by the rack ID acquirer 67 of FIG. 3. In the rack ID acquisition processing, as in Step S103 of FIG. 8A, the target rack ID 26 assigned to the rack number 3 of the rack 2 in which the instrument set 8 is to be stored is acquired. The instrument ID acquisition processing is executed by the sterilization ID acquirer 65 of FIG. 3. In the instrument ID acquisition processing, as in S105 of FIG. 8A, the target sterilization ID 22 that is an example of the target identification ID used to identify the instrument set 8 that is to be stored is acquired. The rack ID extraction processing is executed by the rack ID extractor 81 of FIG. 3. In the rack ID extraction processing, as in Step S1071 of FIG. 8A, the rack ID 134 associated with the sterilization ID 131 that matches the target sterilization ID 22 that is an example of the target identification ID is extracted from the sterilization information 130 (see FIG. 6) and the extracted rack ID 134 is determined as the extracted rack ID 134a. The rack determination processing is executed by the rack determiner 82 of FIG. 3. In the rack determination processing, as in Step S1073 of FIG. 8A, whether the extracted rack ID 134a matches the target rack ID 26 is determined. The wrong rack notification processing is executed by the wrong rack notifier 83 of FIG. 3. In the wrong rack notification processing, as in Step S109 of FIG. 8A, when it is determined in the rack determination processing that the extracted rack ID 134a does not match the target rack ID 26, wrong rack of the instrument set 8 identified from the target sterilization ID 22 is notified.

In this preferred embodiment, as illustrated in FIG. 6, in the sterilization information 130, the rack ID 134 assigned to the rack number 3 of the rack 2 that is a proper storage destination of the instrument set 8 identified from the sterilization ID 131 is associated with the sterilization ID 131. Herein, in storing the instrument set 8 in the storage process 16 of FIG. 2, as in Step S1071 of FIG. 8A, the rack ID 134 corresponding to the target sterilization ID 22 of the instrument set 8 that the operator is going to store is extracted from the sterilization information 130 and the extracted rack ID 134 is determined as the extracted rack ID 134a. Then, as in Step S1073, whether the target rack ID 26 assigned to the rack number 3 of the rack 2 in which the operator is going to store the instrument set 8 and the extracted rack ID 134a match is determined. Herein, if the target rack ID 26 and the extracted rack ID 134a do not match, it is determined that the rack number 3 of the rack 2 in which the operator is going to store the instrument set 8 is not proper and, as in Step S109, wrong rack is notified. The operator is notified of wrong rack, and thus, can notice wrong rack before actually storing the instrument set 8. Accordingly, it can be prevented in advance that the operator erroneously stores the instrument set 8 in a wrong storage destination.

In this preferred embodiment, in the wrong rack notification processing executed by the wrong rack notifier 83, the wrong rack warning screen DP20 (see FIG. 10) including the wrong rack notification message M20 indicating a wrong rack of the instrument set 8 identified from the target sterilization ID 22 is displayed on the display device 50. Thus, the operator can know in advance that the rack number 3 of the rack 2 in which the operator is going to store the instrument set 8 assigned the target sterilization ID 22 is wrong by viewing the wrong rack warning screen DP20 displayed on the display device 50 as illustrated in FIG. 10. Accordingly, the operator can easily visually recognize a wrong rack of the instrument set 8 assigned the target sterilization ID 22.

In this preferred embodiment, as illustrated in FIG. 6, in the sterilization information 130, at least the sterilization ID 131 indicating that sterilization processing has been performed on the instrument set 8 and the sterilization validity term expiration date 137 of sterilization processing performed on the instrument set 8 are associated with one another. The at least one processor 60b (see FIG. 3) is configured or programmed to execute the instrument ID acquisition processing, expiration date extraction processing, date acquisition processing, expiration determination processing, and expiration notification processing. In the instrument ID acquisition processing executed by the sterilization ID acquirer 65 of FIG. 3, as in Step S105 of FIG. 8A, the target sterilization ID 22 assigned to the instrument set 8 is acquired. The expiration date extraction processing is executed by the expiration date extractor 86 of FIG. 3. In the expiration date extraction processing, as in Step S1131 of FIG. 8B, the sterilization validity term expiration date 137 associated with the sterilization ID 131 that matches the target sterilization ID 22 is extracted from the sterilization information 130 (see FIG. 6) and the extracted sterilization validity term expiration date 137 is determined as an extracted sterilization validity term expiration date 137a. The date acquisition processing is executed by the date acquirer 87 of FIG. 3. In the date acquisition processing, as in Step S1133 of FIG. 8B, the current date 139 that is a date of a present day is acquired. The expiration determination processing is executed by the expiration determiner 88 of FIG. 3. In the expiration determination processing, as in Step S1135 of FIG. 8B, whether the extracted sterilization validity term expiration date 137a is before the current date 139 is determined. The expiration notification processing is executed by the expiration notifier 89 of FIG. 3. In the expiration notification processing, as in Step S115 of FIG. 8B, when it is determined in the expiration determination processing that the extracted sterilization validity term expiration date 137a is before the current date 139, expiration of validity of sterilization of the instrument set 8 assigned the target sterilization ID 22 is notified.

In this preferred embodiment, as illustrated in FIG. 6, in the sterilization information 130, the sterilization validity term expiration date 137 of sterilization processing for the instrument set 8 assigned the sterilization ID 131 is associated with the sterilization ID 131. Herein, in storing the instrument set 8 in the storage process 16 of FIG. 2, as in Step S1131 of FIG. 8B, the sterilization validity term expiration date 137 corresponding to the target sterilization ID 22 of the instrument set 8 that the operator is going to store is extracted from the sterilization information 130 (see FIG. 6) and the extracted sterilization validity term expiration date 137 is determined as the extracted sterilization validity term expiration date 137a. If the extracted sterilization validity term expiration date 137a is before the current date 139, it is determined that a term of validity of sterilization processing of the instrument set 8 that the operator is going to store has expired and, as in Step S115 of FIG. 8B, expiration of validity of sterilization is notified. The operator is notified of expiration of validity of sterilization, and thus, can notice that the term of validity of sterilization processing has expired before actually storing the instrument set 8. Accordingly, it can be prevented in advance that the operator stores the instrument set 8 for which the term of validity of sterilization processing has expired.

In this preferred embodiment, in the expiration notification processing executed by the expiration notifier 89 of FIG. 3, the sterilization validity expiration warning screen DP 21 (see FIG. 11B) including the sterilization validity expiration notification message M21 indicating that validity of sterilization for the instrument set 8 assigned the target sterilization ID 22 has expired is displayed on the display device 50. Thus, the operator can know in advance that the term of validity of sterilization processing of the instrument set 8 assigned the target sterilization ID 22 has expired by viewing the sterilization validity expiration warning screen DP 21 displayed on the display device 50. Accordingly, the operator can easily visually recognize that the term of validity of sterilization processing for the instrument set 8 assigned the target sterilization ID 22 has expired.

In this preferred embodiment, the storage destination information according to a preferred embodiment of the present invention is included in the sterilization information 130 (see FIG. 6). The identification ID according to a preferred embodiment of the present invention is the same as the sterilization ID 131 of FIG. 6 and is realized by the sterilization ID 131. The target identification ID according to a preferred embodiment of the present invention is the same as the target sterilization ID 22 of FIG. 1 and is realized by the target sterilization ID 22. Thus, by executing processing of acquiring the target sterilization ID 22 in Step S105 in FIG. 8A once, the rack determination processing of Step S1073 (see FIG. 8A), the wrong rack notification processing of Step S109 (see FIG. 8A), the expiration determination processing of Step S1135, and the expiration notification processing of Step S115 in FIG. 8B can be consecutively executed.

In this preferred embodiment, as illustrated in FIG. 1, the instrument set 8 includes the sterilization-later instrument set 8a on which sterilization processing is performed after the sterilization-later instrument set 8a is dispensed from the storage destination. The ID assigned to the rack number 3a of the sterilization-later rack 2a in which the sterilization-later instrument set 8a is stored is determined as the sterilization-later rack ID 26a. The at least one processor 60b of the storage management device 60 is configured or programmed to execute sterilization-later determination processing. The sterilization-later determination processing is executed by the sterilization-later determiner 90 of FIG. 3. In the sterilization-later determination processing, as in Step S111 of FIG. 8B, whether the extracted rack ID 134a matches the sterilization-later rack ID 26a is determined. When it is determined in the sterilization-later determination processing that the extracted rack ID 134a matches the sterilization-later rack ID 26a, sterilization determination processing, unsterilized notification processing, expiration data extraction processing, date acquisition processing, expiration determination processing, and expiration notification processing are not executed. That is, Step S112, Step S112A, Step S1131, Step S1133, Step S1135, and Step S115 in FIG. 8B are not executed. For the sterilization-later instrument set 8a, sterilization processing is performed after storage and before a surgical operation or a medical examination, and therefore, there is no term of validity of sterilization at the time of stocking. Accordingly, in stocking and storing the sterilization-later instrument set 8a, Step S112, Step S112A, Step S1131, Step S1133, Step S1135, and Step S115 in FIG. 8B are omitted, and therefore, the sterilization-later instrument set 8a can be efficiently processed.

In this preferred embodiment, the at least one processor 60b (see FIG. 3) is configured or programmed to execute the sterilization determination processing and the unsterilized notification processing. The sterilization determination processing is executed by the sterilization determiner 84 of FIG. 3. In the sterilization determination processing, as in Step S112 of FIG. 8B, whether there is the sterilization ID 131 that matches the target sterilization ID 22 in the sterilization information 130 (see FIG. 6) is determined. The unsterilized notification processing is executed by the unsterilized notifier 85 of FIG. 3. In the unsterilized notification processing, as in Step S112A of FIG. 8B, when it is determined in the sterilization determination processing that there is not the sterilization ID 131 that matches the target sterilization ID 22 in the sterilization information 130, it is notified that sterilization processing has not been performed on the instrument set 8 assigned the target sterilization ID 22. Thus, the operator is notified that sterilization processing has not been performed, and thus, can notice that sterilization processing has not been performed before actually storing the instrument set 8. Accordingly, an error in which an operator stores the instrument set 8 on which sterilization processing has not been performed is prevented in advance.

In this preferred embodiment, in the unsterilized notification processing executed by the unsterilized notifier 85 of FIG. 3, the unsterilized warning screen DP22 (see FIG. 11A) including the unsterilized notification message M22 indicating that sterilization processing has not been performed on the instrument set 8 assigned the target sterilization ID 22 is displayed on the display device 50. Thus, the operator can know in advance that sterilization processing has not been performed on the instrument set 8 assigned the target sterilization ID 22 by viewing the unsterilized warning screen DP22 displayed on the display device 50. Accordingly, the operator can easily visually recognize that sterilization processing has not been performed on the instrument set 8 assigned the target sterilization ID 22.

In this preferred embodiment, as illustrated in FIG. 1, the storage management system 100 includes the storage management device 60 for the medical instrument 5, the display device 50, the operation device 55, and the reading device 30. The sterilization identification mark 21 in which the target sterilization ID 22 that is an example of the target identification ID is recorded is assigned to the instrument set 8. The rack identification mark 25 in which the target rack ID 26 is recorded is assigned to the rack number 3 of the rack 2 in which the instrument set 8 is stored. The reading device 30 is operated by the operator and can read the sterilization identification mark 21 and the rack identification mark 25. In the rack ID acquisition processing executed by the rack ID acquirer 67 of FIG. 3, the target rack ID 26 is acquired by the reading device 30 reading the rack identification mark 25. In the instrument ID acquisition processing executed by the sterilization ID acquirer 65 of FIG. 3, the target sterilization ID 22 is acquired by the reading device 30 reading the sterilization identification mark 21. Thus, the operator uses a simple method in which the sterilization identification mark 21 of the instrument set 8 that the operator is going to store is read by the reading device 30, so that the storage management device 60 can acquire the target sterilization ID 22. Moreover, the operator uses a simple method in which the rack identification mark 25 assigned to the rack number 3 of the rack 2 in which the operator is going to store the instrument set 8 is read by the reading device 30, so that the storage management device 60 can acquire the target rack ID 26.

In this preferred embodiment, as illustrated in FIG. 8A, Step S107 that is wrong rack determination processing, Step S111 that is determination processing of determining whether the instrument set 8 is the sterilization-later instrument set 8a, Step S112 that is determination processing of determining whether sterilization processing has been performed (in other words, whether the instrument set 8 is unsterilized), Step S113 that is determination processing of determining whether a term of validity of sterilization has expired, and Step S117 that is determination processing of determining re-stocking are sequentially executed. However, there is no particular limitation on an order in which Step S107, Step S111, Step S112, Step S1113, and Step S117 are executed, and the order of the steps may be switched as appropriate. For example, the steps may be executed in an order of Step S111, Step S112, Step 113, Step S107, and Step 117.

In this preferred embodiment, the identification ID according to a preferred embodiment of the present invention is the sterilization ID 131 of the sterilization information 130. However, the identification ID according to a preferred embodiment of the present invention may be the set ID 132 from which the instrument set 8 can be identified. In this case, the target identification ID according to a preferred embodiment of the present invention may be a target set ID assigned to the instrument set 8.

In this preferred embodiment, the storage destination information and the sterilization information according to a preferred embodiment of the present invention are realized by the sterilization information 130 and are stored in the sterilization label information table TB30. However, the storage destination information and the sterilization information according to a preferred embodiment of the present invention are different information from one another and may be stored in separate tables.

In this preferred embodiment, the storage management method for the medical instrument 5 can be realized by the storage management device 60 for the medical instrument 5. The storage management method for the medical instrument 5 according to this preferred embodiment includes a display step, a sterilization ID acquisition step, a rack ID acquisition step, a determination step, a notification step, a list addition step, a history addition step, a rack ID extract step, a rack determination step, a wrong rack notification step, a sterilization determination step, an unsterilized notification step, an expiration date extraction step, a date acquisition step, an expiration determination step, an expiration notification step, and a sterilization-later determination step. The display step, the sterilization ID acquisition step, the rack ID acquisition step, the determination step, the notification step, the list addition step, the history addition step, the rack ID extract step, the rack determination step, the wrong rack notification step, the sterilization determination step, the unsterilized notification step, the expiration date extraction step, the date acquisition step, the expiration determination step, the expiration notification step, and the sterilization-later determination step are realized by the display section 63, the sterilization ID acquirer 65, the rack ID acquirer 67, the determiner 69, the notifier 71, the list adder 73, the history adder 75, the rack ID extractor 81, the rack determiner 82, the wrong rack notifier 83, the sterilization determiner 84, the unsterilized notifier 85, the expiration date extractor 86, the date acquirer 87, the expiration determiner 88, the expiration notifier 89, and the sterilization-later determiner 90 of the storage management device 60, respectively.

While preferred embodiments of the present invention have been described above, it is to be understood that variations and modifications will be apparent to those skilled in the art without departing from the scope and spirit of the present invention. The scope of the present invention, therefore, is to be determined solely by the following claims.

What is claimed is:

1. A storage management device for a medical instrument,
the storage management device comprising:
a memory; and
at least one processor; wherein
storage destination information in which at least an identification ID used to identify an instrument set including one or more medical instruments and a rack ID assigned to a rack number of a rack in which the instrument set is to be stored are associated with one another is stored; and
the at least one processor is configured to execute a program stored in the memory to perform:
rack ID acquisition processing to acquire a target rack ID assigned to the rack number of the rack in which the instrument set is to be stored;
instrument ID acquisition processing to acquire a target identification ID used to identify the instrument set that is to be stored;
rack ID extraction processing to extract the rack ID associated with the identification ID that matches the target identification ID from the storage destination information to determine the rack ID as an extracted rack ID;
rack determination processing to determine whether the extracted rack ID matches the target rack ID; and
wrong rack notification processing to notify, when it is determined in the rack determination processing that the extracted rack ID does not match the target rack ID, a wrong rack of the instrument set that is identified from the target identification ID.

2. The storage management device for a medical instrument according to claim 1, wherein, in the wrong rack notification processing, a wrong rack warning screen including a wrong rack notification message indicating the wrong rack of the instrument set that is identified from the target identification ID is displayed on a display device.

3. The storage management device for a medical instrument according to claim 1, wherein
sterilization information in which at least a sterilization ID indicating that sterilization processing has been performed on the instrument set and a sterilization validity term expiration date of the sterilization processing that has been performed on the instrument set are associated with one another is stored;
in the instrument ID acquisition processing, a target sterilization ID assigned to the instrument set is acquired;
the at least one processor is configured to execute the program stored in the memory to perform:
expiration date extraction processing to extract the sterilization validity term expiration date associated with the sterilization ID that matches the target sterilization ID from the sterilization information to determine the extracted sterilization validity term expiration date as an extracted sterilization validity term expiration date;
date acquisition processing to acquit a current date;
expiration determination processing to determine whether the extracted sterilization validity term expiration date is before the current date; and
expiration notification processing to notify, when it is determined in the expiration determination processing that the extracted sterilization validity term expiration date is before the current date, expiration of validity of sterilization of the instrument set assigned the target sterilization ID.

4. The storage management device for a medical instrument according to claim 3, wherein, in the expiration notification processing, a sterilization validity expiration warning screen including a sterilization validity expiration notification message indicating that a validity of sterilization of the instrument set assigned the target sterilization ID has expired is displayed on a display device.

5. The storage management device for a medical instrument according to claim 3, wherein
the storage destination information is included in the sterilization information;
the identification ID is a same ID as the sterilization ID; and
the target identification ID is a same ID as the target sterilization ID.

6. The storage management device for a medical instrument according to claim 3, wherein
the instrument sets include a sterilization-later instrument set on which sterilization processing is performed after the instrument set has been dispensed from a storage destination;
the at least one processor is configured to execute the program stored in the memory to:
perform, when an ID assigned to a rack number of a sterilization-later rack in which the sterilization-later instrument set is to be stored is determined as a sterilization-later rack ID, sterilization-later determination processing to determine whether the extracted rack ID matches the sterilization-later rack ID; and
not to perform, when it is determined that in the sterilization-later determination processing, that the extracted rack ID matches the sterilization-later rack ID, the expiration date extraction processing, the date acquisition processing, the expiration determination processing, and the expiration notification processing.

7. The storage management device for a medical instrument according to claim 1, wherein
sterilization information including a sterilization ID indicating that sterilization processing has been performed on the instrument set is stored;
in the instrument ID acquisition processing, a target sterilization ID assigned to the instrument set is acquired; and
the at least one processor is configured to execute the program stored in the memory to perform:
sterilization determination processing to determine whether the sterilization ID that matches the target sterilization ID is included in the sterilization information; and
unsterilized notification processing of notifying, when it is determined in the sterilization determination processing that the sterilization ID that matches the target sterilization ID is not included in the sterilization information, that sterilization processing has not been performed on the instrument set assigned the target sterilization ID.

8. The storage management device for a medical instrument according to claim 7, wherein, in the unsterilized notification processing, an unsterilized warning screen including an unsterilized notification message indicating that sterilization processing has not been performed on the instrument set assigned the target sterilization ID is displayed on a display device.

9. A storage management system comprising:
the storage management device for a medical instrument according to claim 1;
a display device; and
an operation device.

10. The storage management system according to claim 9, further comprising:
a reading device that is operated by an operator to read an identification mark and a rack identification mark; wherein
in the identification mark, the target identification ID is recorded, and the identification mark is assigned to the instrument set;
in the rack identification mark, the target rack ID is recorded, and the rack identification mark is assigned to a rack number of a rack in which the instrument set is to be stored;
in the rack ID acquisition processing, the target rack ID is acquired by reading the rack identification mark by the reading device; and
in the instrument ID acquisition processing, the identification ID is acquired by reading the identification mark by the reading device.

11. A storage management method for a medical instrument,
the storage management method comprising:
storing storage destination information in which at least an identification ID used to identify an instrument set including one or more medical instruments and a rack ID assigned to a rack number of a rack in which the instrument set is to be stored are associated with one another;
a rack ID acquisition step of acquiring a target rack ID assigned to the rack number of the rack in which the instrument set is to be stored;
an instrument identification step of acquiring a target identification ID used to identify the instrument set that is to be stored;
a rack ID extraction step of extracting the rack ID associated with the identification ID that matches the target identification ID from the storage destination information to determine the rack ID as an extracted rack ID;
a rack determination step of determining whether the extracted rack ID matches the target rack ID; and
a wrong rack notification step of notifying, when it is determined in the rack determination processing that the extracted rack ID does not match the target rack ID, a wrong rack of the instrument set that is identified from the target identification ID.

12. The storage management method for a medical instrument according to claim 11, wherein, in the wrong rack notification step, a wrong rack warning screen including a wrong rack notification message indicating the wrong rack of the instrument set that is identified from the target identification ID is displayed on a display device.

13. The storage management method for a medical instrument according to claim 11, wherein
sterilization information in which at least a sterilization ID indicating that sterilization processing has been performed on the instrument set and a sterilization validity term expiration date of the sterilization processing that has been performed on the instrument set are associated with one another is stored;
the storage management method further includes:
acquiring, in the instrument identification step, a target sterilization ID assigned to the instrument set;
an expiration date extraction step of extracting the sterilization validity term expiration date associated with the sterilization ID that matches the target sterilization ID from the sterilization information to determine the sterilization validity term expiration date as an extracted sterilization validity term expiration date;
a date acquisition step of acquiring a current date;
an expiration date determination step of determining whether the extracted sterilization validity term expiration date is before the current date; and
an expiration notification step of notifying, when it is determined in the expiration date determination step that the extracted sterilization validity term expiration date is before the current date, expiration of validity of sterilization of the instrument set assigned the target sterilization ID.

14. The storage management method for a medical instrument according to claim 13, wherein
the instrument sets include a sterilization-later instrument set on which sterilization processing is performed after the instrument set has been dispensed from a storage destination;
the storage management method further includes a sterilization-later determination step of determining whether the extracted rack ID matches a sterilization-later rack ID, when an ID assigned to a rack number of a sterilization-later rack in which the sterilization-later instrument set is to be stored is determined as the sterilization-later rack ID; and
when it is determined that in the sterilization-later determination step that the extracted rack ID matches the sterilization-later rack ID, the expiration date extraction step, the date acquisition step, the expiration date determination step, and the expiration notification step are not executed.

15. The storage management method for a medical instrument according to claim 11, wherein
sterilization information including a sterilization ID indicating that sterilization processing has been performed on the instrument set is stored;
in the instrument identification step, a target sterilization ID assigned to the instrument set is acquired; and
the storage management method further includes:
a sterilization determination step of determining whether the sterilization ID that matches the target sterilization ID is included in the sterilization information; and
an unsterilized notification step of notifying, when it is determined in the sterilization determination step that the sterilization ID that matches the target sterilization ID is not included in the sterilization information, that sterilization processing has not been performed on the instrument set assigned the target sterilization ID.
